(19) United States
(12) Patent Application Publication
(10) Pub. No.: US 2025/0382323 A1
(43) Pub. Date: Dec. 18, 2025

(54) COMPOSITIONS, METHODS, AND SYSTEMS FOR DETECTING NUCLEOTIDES

(71) Applicant: AXBIO INC., Santa Clara, CA (US)

(72) Inventor: Tao HONG, Santa Clara, CA (US)

(21) Appl. No.: 19/170,248

(22) Filed: Apr. 4, 2025

Related U.S. Application Data
(63) Continuation of application No. PCT/US23/76028, filed on Oct. 4, 2023.
(60) Provisional application No. 63/413,305, filed on Oct. 5, 2022.

(51) Int. Cl. C07H 19/20; C12Q 1/6869

(52) U.S. Cl. CPC C07H 19/20; C12Q 1/6869

(57) ABSTRACT

An aspect of the present disclosure provides an engineered nucleotide molecule. The engineered nucleotide molecule can comprise a pentose sugar. The engineered nucleotide molecule can comprise a base coupled to the pentose sugar, wherein the base is selected from the group consisting of adenine, guanine, cytosine, thymine, uracil, and an analogue thereof. The engineered nucleotide molecule can comprise a polyphosphate chain coupled to the pentose sugar. The engineered nucleotide molecule can comprise a protecting group coupled to the pentose sugar, wherein the protecting group is configured to inhibit coupling of an additional nucleotide to the engineered nucleotide molecule. The engineered nucleotide molecule can comprise an identifier moiety coupled to the pentose sugar via the polyphosphate chain, wherein the identifier moiety is specific for the engineered nucleotide molecule.

### CROSS REFERENCE

This application is a continuation of International Patent Application No. PCT/US23/76028, filed Oct. 4, 2023, which claims the benefit of U.S. Provisional Application No. 63/413,305, filed Oct. 5, 2022, each of which is entirely incorporated herein by reference.

### BACKGROUND

Nucleic acid sequencing is the process of determining the sequence of nucleotides in a nucleic acid sample. Specific nucleic acid sequence information can be used in the discovery or identification of genetic diseases, diagnosis of infectious diseases, and development and monitoring of treatment.

A variety of nucleic acid sequencing methods have been investigated, for example, electrophoresis, sequencing by hybridization, mass spectrometry-based method, sequencing by ligation, and sequencing by synthesis (SBS).

### SUMMARY

The present disclosure provides methods and systems for analyzing a sample (e.g., a nucleic acid sample derived from a biological sample).

In an aspect, the present disclosure provides an engineered nucleotide molecule, comprising: a pentose sugar; a base coupled to the pentose sugar, wherein the base is selected from the group consisting of adenine, guanine, cytosine, thymine, uracil, and an analogue thereof; a polyphosphate chain coupled to the pentose sugar, wherein the polyphosphate chain comprises two or more phosphate groups; a protecting group coupled to the pentose sugar, wherein the protecting group is configured to inhibit coupling of an additional nucleotide to the engineered nucleotide molecule; and an identifier moiety coupled to the pentose sugar, wherein the identifier moiety is specific for the engineered nucleotide molecule, wherein the identifier moiety is directly coupled to the polyphosphate chain.

In some embodiments of any one of the engineered nucleotide molecules disclosed herein, the pentose sugar is deoxyribose.

In some embodiments of any one of the engineered nucleotide molecules disclosed herein, the polyphosphate chain comprises three or more phosphate groups. In some embodiments of any one of the engineered nucleotide molecules disclosed herein, the polyphosphate chain comprises four or more phosphate groups. In some embodiments of any one of the engineered nucleotide molecules disclosed herein, the polyphosphate chain comprises six phosphate groups.

In some embodiments of any one of the engineered nucleotide molecules disclosed herein, the hydroxyl group is at the 3′ position of the pentose sugar.

In some embodiments of any one of the engineered nucleotide molecules disclosed herein, the protecting group is coupled to a hydroxyl group of the pentose sugar. In some embodiments of any one of the engineered nucleotide molecules disclosed herein, the protecting group comprises allyl or azide. In some embodiments of any one of the engineered nucleotide molecules disclosed herein, the protecting group is removable from the engineered nucleotide molecule.

In some embodiments of any one of the engineered nucleotide molecules disclosed herein, the identifier moiety is removable from the engineered nucleotide molecule. In some embodiments of any one of the engineered nucleotide molecules disclosed herein, the identifier moiety comprises a polynucleotide. In some embodiments of any one of the engineered nucleotide molecules disclosed herein, the identifier moiety comprises a non-polynucleotide/non-polypeptide polymer.

In some embodiments of any one of the engineered nucleotide molecules disclosed herein, the polynucleotide has a length of at least about 5 bases. In some embodiments of any one of the engineered nucleotide molecules disclosed herein, the polynucleotide has a length of at least about 10 bases. In some embodiments of any one of the engineered nucleotide molecules disclosed herein, the polynucleotide has a length of at least about 20 bases. In some embodiments of any one of the engineered nucleotide molecules disclosed herein, the polynucleotide has a length of at least about 30 bases.

In some embodiments of any one of the engineered nucleotide molecules disclosed herein, the polynucleotide comprises a polyN selected from the group consisting of polyA, polyT, polyC, polyG, polyU, and a variant thereof.

In another aspect, the present disclosure provides a method of analyzing a target nucleic acid molecule, comprising: (a) providing a complex comprising (i) the target nucleic acid molecule and (ii) a primer nucleic acid molecule exhibiting complementarity to a portion of the target nucleic acid molecule; (b) contacting the complex with an engineered nucleotide molecule, to generate a growing strand coupled to the primer nucleic acid molecule, wherein the growing stand exhibits sequence complementarity to an additional portion of the target nucleic acid molecule, and wherein the engineered nucleotide molecule comprises: a pentose sugar; a base coupled to the pentose sugar, wherein the base is selected from the group consisting of adenine, guanine, cytosine, thymine, uracil, and an analogue thereof; a polyphosphate chain coupled to the pentose sugar, wherein the polyphosphate chain comprises two or more phosphate groups; a protecting group coupled to the pentose sugar, wherein the protecting group is configured to inhibit coupling of an additional nucleotide to the engineered nucleotide; and an identifier moiety coupled to the pentose sugar, wherein the identifier moiety is specific for the engineered nucleotide, wherein the identifier moiety is directly coupled to the polyphosphate chain.

In some embodiments of any one of the methods disclosed herein, the method further comprises using a sensor moiety for detection of (i) the contacting or (ii) generation of the growing strand.

In some embodiments of any one of the methods disclosed herein, the detection comprises measuring one or more signals indicative of impedance or impedance change in the sensor moiety upon (i) the contacting or (ii) generation of the growing strand.

In some embodiments of any one of the methods disclosed herein, the method further comprises contacting the complex with the sensor moiety, to incorporate at least a portion of the engineered nucleotide molecule as part of the growing strand.

In some embodiments of any one of the methods disclosed herein, the sensor moiety comprises a pore or an enzyme. In some embodiments of any one of the methods disclosed herein, the sensor moiety comprises the pore and the enzyme coupled to the pore. In some embodiments of any one of the methods disclosed herein, the pore is part of a nanopore protein. In some embodiments of any one of the methods disclosed herein, the pore is part of a solid-state nanopore.

In some embodiments of any one of the methods disclosed herein, the enzyme comprises a polymerase.

In some embodiments of any one of the methods disclosed herein, the method further comprises, subsequent to (b), removing the protecting group from the pentose sugar.

In some embodiments of any one of the methods disclosed herein, the method further comprises, subsequent to the removing, coupling the additional nucleotide to the engineered nucleotide.

In some embodiments of any one of the methods disclosed herein, the removing of the protecting group comprises an enzymatic reaction.

In some embodiments of any one of the methods disclosed herein, the removing of the protecting group comprises an enzyme-free chemical reaction.

In some embodiments of any one of the methods disclosed herein, the method further comprises, subsequent to (b), removing the identifier moiety from the pentose sugar.

In some embodiments of any one of the methods disclosed herein, the pentose sugar is deoxyribose.

In some embodiments of any one of the methods disclosed herein, the polyphosphate chain comprises three or more phosphate groups.

In some embodiments of any one of the methods disclosed herein, the polyphosphate chain comprises four or more phosphate groups.

In some embodiments of any one of the methods disclosed herein, the hydroxyl group is at the 3′ position of the pentose sugar. In some embodiments of any one of the methods disclosed herein, the protecting group is coupled to a hydroxyl group of the pentose sugar. In some embodiments of any one of the methods disclosed herein, the protecting group is removable from the pentose sugar. In some embodiments of any one of the methods disclosed herein, wherein the protecting group comprises allyl or azide.

In some embodiments of any one of the methods disclosed herein, the identifier moiety is removable from the engineered nucleotide molecule.

In some embodiments of any one of the methods disclosed herein, the identifier moiety comprises a polynucleotide sequence that does not exhibit complementarity to at least a portion of the target nucleic acid molecule.

In some embodiments of any one of the methods disclosed herein, the identifier moiety comprises a polynucleotide. In some embodiments of any one of the methods disclosed herein, the identifier moiety comprises a non-polynucleotide/non-polypeptide polymer. In some embodiments of any one of the methods disclosed herein, the polynucleotide has a length of at least about 5 bases. In some embodiments of any one of the methods disclosed herein, the polynucleotide has a length of at least about 10 bases. In some embodiments of any one of the methods disclosed herein, the polynucleotide has a length of at least about 20 bases. In some embodiments of any one of the methods disclosed herein, the polynucleotide has a length of at least about 30 bases. In some embodiments of any one of the methods disclosed herein, the polynucleotide comprises a polyN selected from the group consisting of polyA, polyT, polyC, polyG, polyU, and a variant thereof.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 schematically illustrates an example of an engineered nucleotide molecule, in accordance with some embodiments.

FIG. 2 schematically illustrates another example of an engineered nucleotide molecule, in accordance with some embodiments.

FIG. 3 shows a computer system that is programmed or otherwise configured to implement methods provided herein.

FIG. 4 shows an exemplary method of analyzing a target nucleic acid molecule, in accordance with some embodiments.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used in the specification and claims, the singular forms “a,” “an,” and “the” can include plural references unless the context clearly dictates otherwise. For example, the term “a sequencing sensor” can include a plurality of sequencing sensors.

The terms “about,” and “approximately,” as used interchangeably herein, generally refer to within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, such as within 5-fold or within 2-fold of a value. Where particular values are described, unless otherwise stated, the term “about” can mean within an acceptable error range for the particular value.

The terms “protecting group,” “blocking group,” and “reversible terminator,” as used interchangeably herein, generally refer to any atom or group of atoms that is added to a molecule in order to prevent existing groups in the molecule from undergoing unwanted chemical reactions. For example, to ensure a single incorporation of a complementary nucleotide that is opposite a base of a target nucleic acid molecule that is being sequenced via sequencing-by-synthesis (SBS), a protecting group can be added to an engineered nucleotide molecule (e.g., at the 3′ hydroxy group of the deoxyribose of the engineered nucleotide molecule) that is incorporated to the growing strand. Simultaneously with or subsequent to the incorporation (e.g., via an enzyme, such as a polymerase) of the engineered nucleotide molecule to the growing strand, the protecting group of the engineered nucleotide molecule can be removed (e.g., via an enzymatic reaction, a chemical reaction, an electromagnetic radiation, etc.), under reaction conditions which do not interfere with the integrity of the target nucleic acid molecule being sequenced. The SBS sequencing cycle can continue accordingly with the incorporation of the next engineered nucleotide molecule with a protecting group.

The terms “identifier moiety,” “label,” and “tag,” as used interchangeably herein, generally refer to a directly or indirectly detectable molecule that is conjugated directly or indirectly to a target compound or composition to be detected, e.g., a nucleotide molecule. The identifier moiety may be detectable by itself (e.g., radioisotope labels or fluorescent labels) or, in the case of an enzymatic label, may catalyze chemical alteration of a substrate compound or composition which is detectable. In some cases, presence or absence of the identifier moiety may be detectable by measuring an electrochemical property (e.g., capacitance, resistance, impedance, conductivity, voltage, etc.) of an electrochemical cell (e.g., a nanopore sensor) upon addition or removal of the identifier moiety, respectively. The identifier moiety can be suitable for small scale detection or more suitable for high-throughput screening. As such, non-limiting examples of the identifier moiety may include radioisotopes, fluorochromes, chemiluminescent compounds, bioluminescent compounds, dyes, polynucleotides, polypeptides (e.g., enzymes, fluorescent proteins, etc.), and non-polynucleotide/non-polypeptide polymers. The identifier moiety may be simply detected. Alternatively or in addition to, the identifier moiety may be quantified.

The terms “polynucleotide,” “oligonucleotide,” “oligomer,” and “nucleic acid,” as used interchangeably herein, generally refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be deoxyribonucleic acid (DNA). A polynucleotide can be ribonucleic acid (RNA). A polynucleotide can have any three dimensional structure, and can perform any function. A polynucleotide can comprise one or more analogs (e.g., altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, complementary DNA (cDNA, such as double-strand cDNA (dd-cDNA) or single-stranded cDNA (ss-cDNA)), circulating tumor DNA (ctDNA), damaged DNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes (e.g., fluorescence in situ hybridization (FISH) probes), and primers. The sequence of nucleotides can be interrupted by non-nucleotide components. A polynucleotide may comprise one or more modified nucleotides, such as methylated nucleotides and nucleotide analogs. The sequence of nucleotides may be interrupted by non-nucleotide components. A polynucleotide can be further modified after polymerization, such as by conjugation with a labeling component.

The terms “complement,” “complements,” “complementary,” and “complementarity,” as used interchangeably herein, generally refer to a sequence that is fully complementary to and hybridizable to the given sequence. A sequence hybridized with a given nucleic acid is referred to as the “complement” or “reverse-complement” of the given molecule if its sequence of bases over a given region is capable of complementarily binding those of its binding partner, such that, for example, adenine (A)-thymine (T), A-uracil (U), guanine (G)-cytosine (C), and G-U base pairs are formed. In general, a first sequence that is hybridizable to a second sequence is specifically or selectively hybridizable to the second sequence, such that hybridization to the second sequence or set of second sequences is preferred (e.g., thermodynamically more stable under a given set of conditions, such as stringent conditions commonly used in the art) to hybridize with non-target sequences during a hybridization reaction. Typically, hybridizable sequences share a degree of sequence complementarity over all or a portion of their respective lengths, such as from about 25% to about 100% complementarity, including at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, and 100% sequence complementarity. The respective lengths may comprise a region of at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, or more nucleotides. Sequence identity, such as for the purpose of assessing percent complementarity, can be measured by any suitable alignment algorithm, including but not limited to the Needleman-Wunsch algorithm (see e.g., the EMBOSS Needle aligner available at www.ebi.ac.uk/Tools/psa/emboss_needle/nucleotide.html, optionally with default settings), the BLAST algorithm (see e.g., the BLAST alignment tool available at blast.ncbi.nlm.nih.gov/Blast.cgi, optionally with default settings), or the Smith-Waterman algorithm (see e.g., the EMBOSS Water aligner available at www.ebi.ac.uk/Tools/psa/emboss_water/nucleotide.html, optionally with default settings). Optimal alignment can be assessed using any suitable parameters of a chosen algorithm, including default parameters.

Complementarity can be perfect or substantial/sufficient. Perfect complementarity between two nucleic acids can mean that the two nucleic acids can form a duplex in which every base in the duplex is bonded to a complementary base by Watson-Crick pairing. Substantial or sufficient complementary can mean that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm of hybridized strands, or by empirical determination of Tm by using routine methods.

The term “hybridization” as used herein, generally refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may comprise two strands forming a duplex structure, three or more strands forming a multi-stranded complex, a single self-hybridizing strand, or any combination of these. A hybridization reaction may constitute a step in a more extensive process, such as the initiation of PCR, or the enzymatic cleavage of a polynucleotide by an endonuclease. A second sequence that is complementary to a first sequence may be referred to as the “complement” of the first sequence. The term “hybridizable,” as applied to a polynucleotide, generally refers to the ability of the polynucleotide to form a complex that is stabilized via hydrogen bonding between the bases of the nucleotide residues in a hybridization reaction.

The term “polymerase,” as used herein, generally refers to an enzyme (e.g., natural or synthetic) capable of catalyzing a polymerization reaction. Examples of polymerases can include a nucleic acid polymerase (e.g., a deoxyribonucleic acid (DNA) polymerase or a ribonucleic acid (RNA) polymerase) and a transcriptase (e.g., a reverse transcriptase). A polymerase can be a polymerization enzyme. The term “DNA polymerase” generally refers to an enzyme capable of catalyzing a polymerization reaction of DNA.

The term “sequencing,” as used herein, generally refers to a procedure for determining the order in which nucleotides occur in a target nucleotide sequence. Methods of sequencing can comprise high-throughput sequencing, such as, for example, next-generation sequencing (NGS). Sequencing may be whole-genome sequencing or targeted sequencing. Sequencing may be single molecule sequencing or massively parallel sequencing. Next-generation sequencing methods can be useful in obtaining millions of sequences in a single run. In an example, sequencing may be performed using one or more nanopore sequencing methods, e.g., sequencing-by-synthesis, sequencing-by-ligation, or sequencing-by-cleavage.

The term “nanopore,” as used herein, generally refers to a pore, channel, or passage formed or otherwise provided in a membrane. The membrane may be an organic membrane, such as a lipid bilayer, or a synthetic membrane, such as a membrane formed of a polymeric material such as a protein nanopore. The membrane may be a solid-state membrane (e.g., silicon substrate). The nanopore may be disposed adjacent or in proximity to a sensing circuit or an electrode coupled to a sensing circuit, such as, for example, a complementary metal-oxide semiconductor (CMOS) or field effect transistor (FET) circuit. The nanopore may be part of the sensing circuit. A nanopore can have a characteristic width or diameter, for example, on the order of about 0.1 nanometer (nm) to 1000 nm. A nanopore can be a biological nanopore, solid state nanopore, hybrid biological solid state nanopore, a variation thereof, or a combination thereof. Examples of the biological nanopore include, but are not limited to, OmpG from E. coli, sp., Salmonella sp., Shigella sp., and Pseudomonas sp., and alpha hemolysin (α-hemolysin) from S. aureus sp., MspA from M. smegmatis sp, a functional variant thereof, or a combination thereof. Sequencing may comprise forward sequencing and/or reverse sequencing. Examples of the solid state nanopore include, but are not limited to, silicon nitride, silicon oxide, graphene, molybdenum sulfide, a functional variant thereof, or a combination thereof. The solid state nanopore may be fabricated by high-energy beam manufacturing, imprinting (e.g., nanoimprinting), laser ablation, chemical etching, plasma etching (e.g., oxygen plasma etching), etc.

The terms “nanopore sequencing,” and “nanopore-based sequencing,” as used interchangeably herein, generally refer to a method that determines the sequence of a polynucleotide with the aid of a nanopore. In some cases, the sequence of the polynucleotide may be determined in a template-dependent manner.

The terms “real-time,” and “real time,” as used interchangeably herein, generally refer to an event (e.g., an operation, a process, a measurement, a detection, etc.) that is performed almost immediately after or within a short period of time after another event (e.g., addition of a nucleobase, generation of a growing strand, etc.), such as within at least about 0.0001millisecond (ms), at least about 0.0005 ms, at least about 0.001 ms, at least about 0.005 ms, at least about 0.01 ms, at least about 0.05 ms, at least about 0.1 ms, at least about 0.5 ms, at least about 1 ms, at least about 5 ms, at least about 0.01 seconds, at least about 0.05 seconds, at least about 0.1 seconds, at least about 0.5 seconds, at least about 1 second, or more. In some cases, a real time event may be performed almost immediately after or within a short period of time after another event, such as within at most about 1 second, at most about 0.5 seconds, at most about 0.1 seconds, at most about 0.05 seconds, at most about 0.01 seconds, at most about 5 ms, at most about 1 ms, at most about 0.5 ms, at most about 0.1 ms, at most about 0.05 ms, at most about 0.01 ms, at most about 0.005 ms, at most about 0.001 ms, at most about 0.0005 ms, at most about 0.0001 ms, or less.

The term “sample,” as used herein, generally refers to any sample that may include one or more constituents (e.g., nucleic acid molecules) for processing or analysis. The sample may be a biological sample. The sample may be a cellular or tissue sample. The sample may be a cell-free sample, such as blood (e.g., whole blood), plasma, serum, sweat, saliva, or urine. The sample may be obtained in vivo or cultured in vitro.

The term “substituted” refers to a functional group as described herein such as an alkyl, or a hydrocarbyl, in which at least one bond to a hydrogen atom contained therein is replaced by a bond to non-hydrogen or non-carbon atom, provided that normal valencies are maintained and that the substitution(s) result(s) in a stable compound. Substituted groups also include groups in which one or more bonds to a carbon(s) or hydrogen(s) atom are replaced by one or more bonds, including double or triple bonds, to a heteroatom. Non-limiting examples of substituents include the functional groups described herein, and for example, N, e.g., so as to form —CN.

Reversible termination sequencing technology is a SBS approach that detects the sequence of a nucleic acid template by stepwise elongation of the growing nucleic acid strand. Reversible termination sequencing can comprise modification of a nucleotide molecule with (i) a removable tag, e.g., a fluorescent tag, to the base of the nucleotide molecule through a linker and/or (ii) a reversible terminator (e.g., a protecting group) to 3′ O position of the sugar. However, such modification(s) on the base can leave behind a vestige after cleavage of at least a portion of the linker carrying the tag, which can interfere with the current polymerization step or any subsequent polymerization steps and/or can result in a short read length. In some examples, the cleavage of the tag may not be complete thus leaving residual tag on the growing nucleic acid strand, which may cause background noise in detecting signals from the subsequently added nucleotide molecules, especially in consensus sequencing. Thus, recognized herein is an unmet need for an engineered nucleotide molecule comprising a detectable tag for reversible termination sequencing, wherein the engineered nucleotide molecule can become substantially (e.g., completely) free of (i) the detectable tag and (ii) any linker utilized for joining the detectable tag to the engineered nucleotide molecule, upon incorporation of a portion of the engineered nucleotide molecule (e.g., a sugar coupled to a base) to a polynucleotide sequence (e.g., a growing strand generated by a polymerase during SBS).

Various aspects of the present disclosure provide an engineered nucleotide molecule, compositions thereof, and methods of use thereof, wherein the engineered nucleotide molecule has a protecting group and an identifier moiety. In some embodiments, the engineered nucleotide molecule can comprise protecting group coupled to a sugar (e.g., pentose sugar) of the engineered nucleotide molecule (e.g., at the 3′ O position of the sugar) and an identifier moiety linked to a polyphosphate chain of the engineered nucleotide molecule, to effect incorporation of just one of the engineered nucleotide molecules and detection of signal from one engineered nucleotide molecule in each cycle (e.g., during SBS). In some embodiments, for the incorporation of the engineered nucleotide molecule to a growing strand (or a primer nucleic acid molecule), a 3′ OH of the growing strand can attack the α-phosphate of the polyphosphate chain of the engineered nucleotide molecule to be incorporated, resulting in a phosphodiester linkage and the release of the other polyphosphate groups containing the identifier moiety. Thus, having the identifier moiety linked to the phosphate that is to be released during the polymerization step (e.g., naturally released by the same mechanism of the polymerization step) can enhance sequencing or may not disrupt sequencing by, e.g., (i) having substantially no vestige left on the remainder of the engineered nucleotide molecule, and/or (ii) having substantially no residual identifier moiety on the growing strand. Thus, any signal detected during incorporation of an engineered nucleotide molecule may only be attributed to the newly added engineered nucleotide molecule, and not to any previously added nucleobases.

### I. Engineered Nucleotide Molecule

In an aspect, the present disclosure provides an engineered nucleotide molecule, a composition thereof, a method of use thereof (e.g., for sequencing a target nucleic acid molecule), and a system for analyzing a target nucleic acid molecule. The engineered nucleotide molecule can comprise a sugar (e.g., a pentose sugar), a base coupled to the sugar, a polyphosphate chain coupled to the sugar, a protecting group coupled to the sugar, and an identifier moiety coupled to the sugar. The identifier moiety can be coupled to the sugar via the polyphosphate chain. Alternatively, the identifier moiety can be coupled to a different portion of the engineered nucleotide molecule (e.g., to the base).

In some embodiments, the sugar can be, for example, pentose, hexose, glucose, fructose, or galactose. In some embodiments, the sugar can be a pentose sugar, for example, ribose, deoxyribose, arabinofuranose, lyxofuranose, or xylofuranose. In some embodiments, the pentose sugar can be ribose (e.g., for a growing RNA strand). In some embodiments, the pentose sugar can be deoxyribose (e.g., for a growing DNA strand).

In some embodiments, the base can be selected from the group consisting of adenine (A), guanine (G), cytosine (C), thymine (T), uracil (U), and an analogue thereof. Non-limiting examples of such base analogue can include 5-aza-uracil, 2-thio-5-aza-uracil, 2-thio-uracil, 5-hydroxy-uracil, 3-methyl-uracil, 5-carboxymethyl-uracil, 5-propynyl-uracil, 5-taurinomethyl-uracil, 5-taurinomethyl-2-thio-uracil, 1-taurinomethyl-4-thio-uracil, 5-methyl-uracil, dihydrouracil, 2-thio-dihydro-uracil, 5-bromouracil, 2-methoxy-uracil, 2-methoxy-4-thio-uracil, 5-aza-cytosine, 3-methyl-cytosine, N4-acetyl-cytosine, 5-formyl-cytosine, N4-methyl-cytosine, 5-hydroxymethyl-cytosine, pyrrolo-cytosine, 2-thio-cytosine, 2-thio-5-methyl-cytosine, 2-methoxy-cytosine, 2-methoxy-5-methyl-cytosine, hypoxanthine, 1-methyl-hypoxanthine, 7-methyl-hypoxanthine, deazahypoxanthine, 7-deaza-guanine, 7-deaza-8-aza-guanine, 6-thio-guanine, 6-thio-7-deaza-guanine, 6-thio-7-deaza-8-aza-guanine, 7-methyl-guanine, 6-thio-7-methyl-guanine, 6-methoxy-guanine, 1-methyl-guanine, N2-methyl-guanine, N2,N2-dimethyl-guanine, 8-oxo-guanine, 7-methyl-8-oxo-guanine, 1-methyl-6-thio-guanine, N2-methyl-6-thio-guanine, N2,N2-dimethyl-6-thio-guanine, 2-aminopurine, 2, 6-diaminopurine, 7-deaza-adenine, 7-deaza-8-aza-adenine, 2-aminoadenine, 7-deaza-2-aminopurine, 7-deaza-8-aza-2-aminopurine, 7-deaza-2,6-diaminopurine, 6-mercaptopurine, 7-deaza-8-aza-2,6-diaminopurine, 1-methyl-adenine, N6-methyl-adenine, N6-isopentenyl-adenine, N6-(cis-hydroxyisopentenyl)-adenine, 2-methylthio-N6-(cis-hydroxyisopentenyl)-adenine, N6-glycinylcarbamoyl-adenine, N6-threonylcarbamoyl-adenine, 2-methylthio-N6-threonyl carbamoyl-adenine, N6,N6-dimethyl-adenine, 7-methyl-adenine, 2-methylthio-adenine, 2-methoxy-adenine, 4-O-ethylthymine, pyrazolopyrimidine, and any substituted analogue thereof.

In some embodiments, the protecting group can be coupled via a hydroxyl group (or a hydroxy group) of the pentose sugar. In some embodiments, the hydroxyl group can be at the 3′ position on the pentose sugar. Alternatively or in addition to, the hydroxyl group can be at the 2′ position of the pentose sugar (e.g., for a ribose sugar). The protecting group can be any suitable group that can couple to the pentose sugar and can be cleaved by any suitable reaction to regenerate the hydroxyl group. Non-limiting examples of the protecting group can comprise allyl, azide, azo, amine, cyanoethyl, dimethylethyl, dimethylacetamidine, azidomethyl, phenoxyacetyl, alkyldithiomethyl, methoxyacetyl, acetyl, p-toluene sulfonate, phosphate, nitrate, 4-methoxy tetrahydrothiopyranyl, tetrahydrothiopyranyl, 4-methoxy tetrahydrothiopyranyl, tetrahydrothiopyranyl, 5-methyl tetrahydrofuranyl, 5-methyl tetrahydropyranyl, tetrahydropyranyl, tetrahydrofuranyl, methoxytetrahydropyranyl, 2-nitrobenzyl, or any substituted analogue thereof. When the engineered nucleotide molecule is coupled to a growing nucleic acid strand, the protecting group can inhibit coupling of an additional nucleotide to the growing nucleic acid strand.

In some embodiments, the protecting group of a terminal engineered nucleotide molecule on a nucleic acid strand may be cleaved, thereby regenerating a hydroxyl group on the engineered nucleotide molecule (e.g., on the sugar of the engineered nucleotide molecule) to allow subsequent addition of another nucleotide molecule (e.g., another engineered nucleotide molecule as disclosed herein) to the growing nucleic acid strand. The protecting group may be cleaved by any suitable reaction, for example, an enzymatic reaction (e.g., by Bacillus stearothermophilus DNA polymerase I), an enzyme-free chemical reaction (e.g., with phosphine, sodium dithionite, palladium catalyzed reaction,), thermal reaction (e.g., in a polymerase chain reaction (PCR) buffer containing 50 mM KCl, 1.5 mM MgCl₂, 20 mM Tris (pH 8.4 at 25° C.)), or photo cleaving reaction (e.g., upon exposure to an electromagnetic radiation, such as ultraviolet (UV) light).

In some embodiments, the protecting group of the hydroxyl group of the pentose sugar (e.g., that of the 3′-OH of the deoxyribose) of the engineered nucleotide molecule as disclosed herein can be cleaved by an enzyme that is different than the polymerase that effects extension (e.g., polymerization) of the growing nucleic acid strand. Alternatively, the protecting group can be cleaved by the same polymerase that effects the extension.

In some embodiments, the identifier moiety of the engineered nucleotide molecule as disclosed herein can have a size that is sufficiently large to induce a change in the electrochemical property (e.g., capacitance, resistance, impedance, conductivity, voltage, etc.) of the electrochemical cell (e.g., a nanopore sensor, a sensor without a nanopore, etc.) as disclosed herein, when the engineered polynucleotide molecule is sufficiently close to a sensor moiety of the electrochemical cell. In some cases, the change in the electrochemical property can occur and can be detectable prior to, during, or subsequent to release of the identifier moiety from the engineered polynucleotide molecule. For example, the engineered nucleotide molecule can be brought to the nanopore sensor, e.g., via the polymerase that is extending the growing nucleic acid strand, and such complexation of the engineered nucleotide molecule to the polymerase, the growing nucleic acid strand, and/or the target nucleic acid molecule to be analyzed may be sufficient to induce the change in the electrochemical property (e.g., change in capacitance of the nanopore sensor). Thus, in some cases, because the size of the identifier moiety may be responsible for the detection or analysis of the target nucleic acid molecule, the identifier moiety may not need to be a fluorescent molecule.

In some embodiments, the identifier moiety may comprise a polynucleotide sequence that does not exhibit complementarity to at least a portion of the target nucleic acid molecule. In some embodiments, the polynucleotide sequence can exhibit less than or equal to about 90%, less than or equal to about 80%, less than or equal to about 70%, less than or equal to about 60%, less than or equal to about 50%, less than or equal to about 40%, less than or equal to about 30%, less than or equal to about 20%, less than or equal to about 10%, less than or equal to about 9%, less than or equal to about 8%, less than or equal to about 7%, less than or equal to about 6%, less than or equal to about 5%, less than or equal to about 4%, less than or equal to about 3%, less than or equal to about 2%, less than or equal to about 1%, less than or equal to about 0.5%, or less than or equal to about 0.1% sequence identity to the polynucleotide sequence of the target nucleic acid molecule.

The polynucleotide sequence of the identifier moiety can have a length of at least about 5 bases, at least about 10 bases, at least about 15 bases, at least about 20 bases, at least about 25 bases, at least about 30 bases, at least about 35 bases, at least about 40 bases, at least about 45 bases, at least about 50 bases, at least about 55 bases, at least about 60 bases, at least about 65 bases, at least about 70 bases, at least about 75 bases, at least about 80 bases, at least about 85 bases, at least about 90 bases, at least about 95 bases, at least about 100 bases, at least about 110 bases, at least about 120 bases, at least about 130 bases, at least about 140 bases, at least about 150 bases, at least about 160 bases, at least about 170 bases, at least about 180 bases, at least about 190 bases, at least about 200 bases, or more. The length of the polynucleotide sequence of the identifier moiety can be at most about 200 bases, at most about 190 bases, at most about 180 bases, at most about 170 bases, at most about 160 bases, at most about 150 bases, at most about 140 bases, at most about 130 bases, at most about 120 bases, at most about 110 bases, at most about 100 bases, at most about 95 bases, at most about 90 bases, at most about 85 bases, at most about 80 bases, at most about 75 bases, at most about 70 bases, at most about 65 bases, at most about 60 bases, at most about 55 bases, at most about 50 bases, at most about 45 bases, at most about 40 bases, at most about 35 bases, at most about 30 bases, at most about 25 bases, at most about 20 bases, at most about 15 bases, at most about 10 bases, at most about 5 bases, or less.

In some embodiments, the polynucleotide sequence of the identifier moiety can comprise a polyN (e.g., T40, A40, A10, or T10). The polyN can be characterized by having (i) two or more of a same base (e.g., TTTT) or (ii) two or more of a same set of bases (e.g., a polydinucleotide, such as ATATATAT) that are contiguous. The same set of bases can comprise at least two different bases, at least three different bases, at least four different bases, at least five different bases, or more. The same set of bases can comprise at most five different bases, at most four different bases, at most three different bases, or at most two different bases. A length of the same set of bases can be at least about 2 bases, at least about 3 bases, at least about 4 bases, at least about 5 bases, at least about 6 bases, at least about 7 bases, at least about 8 bases, at least about 9 bases, at least about 10 bases, or more. The length of the same set of bases can be at most about 10 bases, at most about 9 bases, at most about 8 bases, at most about 7 bases, at most about 6 bases, at most about 5 bases, at most about 4 bases, at most about 3 bases, or at most about 2 bases. Non-limiting examples of the polyN can comprise polyA, polyT, polyC, polyG, polyU, or poly-dinucleotide (e.g., polyAT, polyCG, polyAG, polyCT, polyAC, polyTG, polyAU). The polyN can have a length of at least about 5 bases, at least about 10 bases, at least about 15 bases, at least about 20 bases, at least about 25 bases, at least about 30 bases, at least about 35 bases, at least about 40 bases, at least about 45 bases, at least about 50 bases, at least about 55 bases, at least about 60 bases, at least about 65 bases, at least about 70 bases, at least about 75 bases, at least about 80 bases, at least about 85 bases, at least about 90 bases, at least about 95 bases, at least about 100 bases, at least about 110 bases, at least about 120 bases, at least about 130 bases, at least about 140 bases, at least about 150 bases, at least about 160 bases, at least about 170 bases, at least about 180 bases, at least about 190 bases, at least about 200bases, or more. The polyN can have a length of at most about 200 bases, at most about 190 bases, at most about 180 bases, at most about 170 bases, at most about 160 bases, at most about 150 bases, at most about 140 bases, at most about 130 bases, at most about 120 bases, at most about 110 bases, at most about 100 bases, at most about 95 bases, at most about 90 bases, at most about 85 bases, at most about 80 bases, at most about 75 bases, at most about 70 bases, at most about 65 bases, at most about 60 bases, at most about 55 bases, at most about 50 bases, at most about 45 bases, at most about 40 bases, at most about 35 bases, at most about 30 bases, at most about 25 bases, at most about 20 bases, at most about 15 bases, at most about 10 bases, at most about 5 bases, or less.

In some embodiments, the identifier moiety can comprise radioactive isotopes, fluorescent labels, chemiluminescent labels, bioluminescent labels and enzyme labels. Non-limiting examples of an identifier moiety (e.g., a fluorescent label) may include fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS), [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP.

In some embodiments, the identifier moiety can comprise polymers that are not polypeptide or polynucleotide. In some embodiment, the polymers are substantially soluble in aqueous conditions. Non-limiting examples of polymers (e.g., a polymer chain or a portion thereof that does not comprise a polynucleotide sequence or a polypeptide sequence) comprise polyethylene glycol, polyethylenimine, polyacrylamide, polyacrylic acid, polyvinyl alcohol, or ionic polymers. In some embodiments, the polymers can be homopolymers. In some embodiments, the polymers can be copolymers.

In some embodiments, the molecular weight of the identifier moiety can be from about 50 dalton (Da) to about 500 Da, from about 50 Da to about 1 kilodalton (kDa), from about 50 Da to about 2 kDa, from about 50 Da to about 5 kDa, from about 50 Da to about 10 kDa, from about 50 Da to about 15 kDa, from about 50 Da to about 20 kDa, from about 50 Da to about 25 kDa, from about 50 Da to about 30 kDa, from about 50 Da to about 35 kDa, from about 50 Da to about 40 kDa, from about 50 Da to about 50 kDa, from about 50 Da to about 60 kDa, from about 50 Da to about 70 kDa, from about 50 Da to about 80 kDa, from about 50 Da to about 90 kDa, from about 50 Da to about 100 kDa, from about 100 Da to about 10 kDa, from about 100 Da to about 15 kDa, from about 100 Da to about 20 kDa, from about 100 Da to about 25 kDa, from about 100 Da to about 30 kDa, from about 100 Da to about 35 kDa, from about 100 Da to about 40 kDa, from about 100 Da to about 50 kDa, from about 100 Da to about 60 kDa, from about 100 Da to about 70 kDa, from about 100 Da to about 80 kDa, from about 100 Da to about 90 kDa, from about 100 Da to about 100 kDa, from about 200 Da to about 10 kDa, from about 200 Da to about 15 kDa, from about 200 Da to about 20 kDa, from about 200 Da to about 25 kDa, from about 200 Da to about 30 kDa, from about 200 Da to about 35 kDa, from about 200 Da to about 40 kDa, from about 200 Da to about 50 kDa, from about 200 Da to about 60 kDa, from about 200 Da to about 70 kDa, from about 200 Da to about 80 kDa, from about 200 Da to about 90 kDa, from about 200 Da to about 100 kDa, from about 500 Da to about 10 kDa, from about 500 Da to about 15 kDa, from about 500 Da to about 20 kDa, from about 500 Da to about 25 kDa, from about 500 Da to about 30 kDa, from about 500 Da to about 35 kDa, from about 500 Da to about 40 kDa, from about 500 Da to about 50 kDa, from about 500 Da to about 60 kDa, from about 500 Da to about 70 kDa, from about 500 Da to about 80 kDa, from about 500 Da to about 90 kDa, from about 500 Da to about 100 kDa, from about 1 kDa to about 10 kDa, from about 1 kDa to about 15 kDa, from about 1 kDa to about 20 kDa, from about 1 kDa to about 25 kDa, from about 1 kDa to about 30 kDa, from about 1 kDa to about 35 kDa, from about 1 kDa to about 40 kDa, from about 1 kDa to about 50 kDa, from about 1 kDa to about 60 kDa, from about 1 kDa to about 70 kDa, from about 1 kDa to about 80 kDa, from about 1 kDa to about 90 kDa, from about 1 kDa to about 100 kDa, from about 2 kDa to about 10 kDa, from about 2 kDa to about 15 kDa, from about 2 kDa to about 20 kDa, from about 2 kDa to about 25 kDa, from about 2 kDa to about 30 kDa, from about 2 kDa to about 35 kDa, from about 2 kDa to about 40 kDa, from about 2 kDa to about 50 kDa, from about 2 kDa to about 60 kDa, from about 2 kDa to about 70 kDa, from about 2 kDa to about 80 kDa, from about 2 kDa to about 90 kDa, from about 2 kDa to about 100 kDa, from about 5 kDa to about 10 kDa, from about 5 kDa to about 15 kDa, from about 5 kDa to about 20 kDa, from about 5 kDa to about 25 kDa, from about 5 kDa to about 30 kDa, from about 5 kDa to about 35 kDa, from about 5 kDa to about 40 kDa, from about 5 kDa to about 50 kDa, from about 5 kDa to about 60 kDa, from about 5 kDa to about 70 kDa, from about 5 kDa to about 80 kDa, from about 5 kDa to about 90 kDa, from about 5 kDa to about 100 kDa, from about 10 kDa to about 15 kDa, from about 10 kDa to about 20 kDa, from about 10 kDa to about 25 kDa, from about 10 kDa to about 30 kDa, from about 10 kDa to about 35 kDa, from about 10 kDa to about 40 kDa, from about 10 kDa to about 50 kDa, from about 10 kDa to about 60 kDa, from about 10 kDa to about 70 kDa, from about 10 kDa to about 80 kDa, from about 10 kDa to about 90 kDa, from about 10 kDa to about 100 kDa, from about 1 kDa to about 25 kDa, from about 20 kDa to about 30 kDa, from about 20 kDa to about 35 kDa, from about 20 kDa to about 40 kDa, from about 20 kDa to about 50 kDa, from about 20 kDa to about 60 kDa, from about 20 kDa to about 70 kDa, from about 20 kDa to about 80 kDa, from about 20 kDa to about 90 kDa, from about 20 kDa to about 100 kDa, from about 30 kDa to about 40 kDa, from about 30 kDa to about 50 kDa, from about 30 kDa to about 60 kDa, from about 30 kDa to about 70 kDa, from about 30 kDa to about 80 kDa, from about 30 kDa to about 90 kDa, from about 30 kDa to about 100 kDa, from about 50 kDa to about 60 kDa, from about 50 kDa to about 70 kDa, from about 50 kDa to about 80 kDa, from about 50 kDa to about 90 kDa, from about 50 kDa to about 100 kDa, from about 60 kDa to about 70 kDa, from about 60 kDa to about 80 kDa, from about 60 kDa to about 90 kDa, from about 60 kDa to about 100 kDa, from about 70 kDa to about 80 kDa, from about 70 kDa to about 90 kDa, from about 70 kDa to about 100 kDa, from about 80 kDa to about 90 kDa, from about 80 kDa to about 100 kDa, and from about 90 kDa to about 100 kDa.

In some embodiments, the engineered nucleotide molecule can initially comprise the identifier moiety, e.g., directly coupled to at least an additional portion of the engineered nucleotide molecule, such as one of the phosphate groups on the polyphosphate chain. In some cases, the identifier moiety can be coupled to the additional portion of the engineered nucleotide molecule via a linker. In some cases, when the identifier moiety is cleaved off from the engineered nucleotide molecule (e.g., during polymerization), at least about 80%, at least about 85%, at least about 90%, at least about 91%, at least about 92%, at least about 93%, at least about 94%, at least about 95%, at least about 96%, at least about 97%, at least about 98%, at least about 99%, or substantially about 100% of the identifier moiety or a combination of the identifier moiety and the linker (e.g., as measured by the molecular weight) can be cleaved or removed from the engineered nucleotide molecule, thereby leaving behind at most about 20%, at most about 15%, at most about 10%, at most about 9%, at most about 8%, at most about 7%, at most about 6%, at most about 5%, at most about 4%, at most about 3%, at most about 2%, at most about 1%, or substantially about 0% of the identifier moiety or the combination of the identifier moiety and the linker in the engineered nucleotide molecule.

In some embodiments, the identifier moiety as disclosed herein can be coupled to a phosphate of the polyphosphate chain via a linker moiety. The linker moiety can be hydrophobic or hydrophilic. Non-limiting examples of the linker moiety include an ester, ether, thioether, ethylene glycol, alkylene, alkenylene, alkynylene, heteroalkylene, cycloalkylene, heterocyclylene, arylene, heteroarylene, and heterocycloalkylene group, any of which can be substituted or unsubstituted. Alternatively, the identifier moiety can be directly coupled to the phosphate of the polyphosphate chain without a separate linker.

In some embodiments, a length of the polyphosphate chain can be at least about 2 phosphates, at least about 3 phosphates, at least about 4 phosphates, at least about 5 phosphates, at least about 6 phosphates, at least about 7 phosphates, at least about 8 phosphates, at least about 9 phosphates, at least about 10 phosphates, at least about 15 phosphates, at least about 20 phosphates, or more. The length of the polyphosphate chain can be at most about 20 phosphates, at most about 15 phosphates, at most about 10 phosphates, at most about 9 phosphates, at most about 8 phosphates, at most about 7 phosphates, at most about 6 phosphates, at most about 5 phosphates, at most about 4 phosphates, or at most about 3 phosphates.

In some embodiments, an engineered nucleotide molecule can comprise a single phosphate group or moiety (e.g., not a polyphosphate chain) coupled to the pentose sugar, and the identifier moiety can directly couple to the single phosphate moiety. In some cases, such engineered nucleotide molecule can be sufficient to facilitate a polymerization reaction in which the identifier moiety is cleaved off and at least the pentose sugar coupled to the base is added to a growing nucleic site, e.g., a growing nucleic acid strand.

In some embodiments, the polyphosphate chain can comprise at least (i) a first phosphate (e.g., an alpha-phosphate or α-phosphate) that is closest to the sugar of the engineered nucleotide molecule and (ii) a second phosphate (e.g., a beta-phosphate or β-phosphate) that is the second closest to the sugar and is directly coupled to the α-phosphate. The identifier moiety can be coupled to (e.g., directly conjugated to) the β-phosphate or any subsequent phosphate group that is coupled thereto. For example, a subsequent phosphate group can be a third phosphate (e.g., a gamma-phosphate or γ-phosphate). In some embodiments, an identifier moiety can be coupled to a terminal phosphate group of the polyphosphate chain (e.g., to γ-phosphate of a triphosphate). Alternatively or in addition to, an identifier moiety can be coupled to a non-terminal phosphate group of the polyphosphate chain (e.g., to β-phosphate of a triphosphate). At least one identifier moiety can be coupled to one of the phosphate groups of the polyphosphate chain as disclosed herein, and the one of the phosphate groups can comprise the β-phosphate, the γ-phosphate, a delta phosphate (or a phosphate at position 4), a epsilon phosphate (or a phosphate at position 5), a zeta phosphate (or a phosphate at position 6), an eta phosphate (or a phosphate at position 7), a theta phosphate (or a phosphate at position 8), an iota phosphate (or a phosphate at position 9), a kappa phosphate (or a phosphate at position 10), a phosphate at position 10, a phosphate at position 11, a phosphate at position 12, a phosphate at position 13, a phosphate at position 14, a phosphate at position 15, a phosphate at position 20, or any subsequent phosphate when available.

In some embodiments, at least the identifier moiety that can be released (e.g., cleaved) from the engineered nucleotide molecule can be detectable, e.g., by a sensor moiety as disclosed herein (e.g., a nanopore sensor). For example, subsequent to the release, detection of the released identifier moiety (e.g., when it is in the vicinity of the nanopore or via entry into the nanopore sensor) can be usable to determine completion of the incorporation of the engineered nucleotide molecule to the growing strand. Alternatively, a separate detection of the released identifier moiety (e.g., other than any measurement thereof during incorporation of the engineered nucleotide molecule into the growing strand) may not be required for accurate detection (e.g., sequence calling) of such incorporation.

In an aspect, the present disclosure provides a method of analyzing a target nucleic acid molecule using an engineered nucleotide molecule and a sensor moiety (e.g., sequencing sensor).

In some embodiments, the method of analyzing a target nucleic acid molecule, comprises a) providing a complex comprising a target nucleic acid molecule and a primer nucleic acid molecule exhibiting complementarity to a portion of the target nucleic acid molecule; and b) contacting the complex with an engineered nucleotide molecule, to generate a growing strand coupled to the primer nucleic acid molecule, wherein the growing stand exhibits sequence complementarity to an additional portion of the target nucleic acid molecule, and wherein the engineered nucleotide molecule comprises a pentose sugar, a base coupled to the pentose sugar, a polyphosphate chain coupled to the pentose sugar, a protecting group coupled to the pentose sugar, and an identifier moiety coupled to the pentose sugar.

In some embodiments, the method further comprises (c) using a sensor moiety to obtain sequence information of at least a portion of the growing strand, to analyze the additional portion of the target nucleic acid molecule.

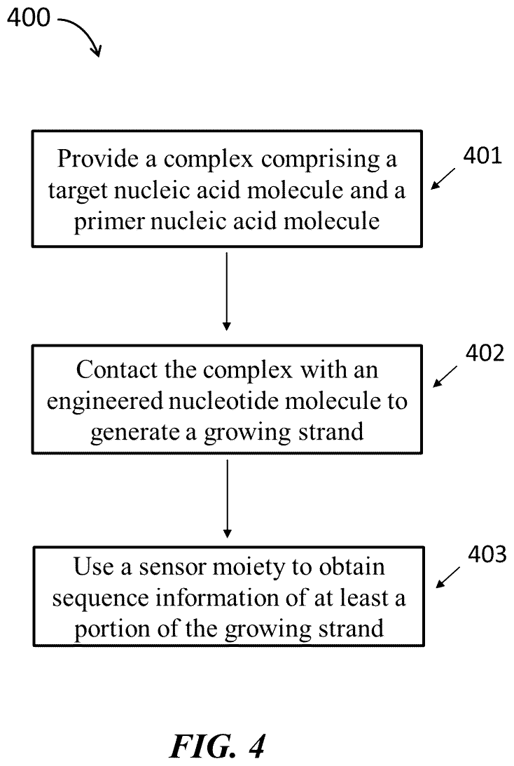

FIG. 4 shows an exemplary method of analyzing a target nucleic acid molecule. At operation 401, the method 400 comprises providing a complex comprising a target nucleic acid molecule and a primer nucleic acid molecule. At operation 402, the method 400 comprises contacting the complex with an engineered nucleotide molecule to generate a growing strand. At operation 403, the method 400 comprises using a sensor moiety to obtain sequence information of at least a portion of the growing strand.

In some embodiments, the engineered nucleotide molecule may comprise (i) a first type of an engineered nucleotide molecule comprising a first type of identifier moiety bound to the pentose sugar via a first type of linker; (ii) a second type of an engineered nucleotide molecule comprising a second type of identifier moiety bound to the pentose sugar via a second type of linker; (iii) a third type of an engineered nucleotide molecule comprising a third type of identifier moiety bound to the pentose sugar via a third type of linker; and (iv) a fourth type of an engineered nucleotide molecule comprising a fourth type of identifier moiety bound to the pentose sugar via a fourth type of linker.

In some embodiments, the first type of identifier moiety, the second type of identifier moiety, the third type of identifier moiety, and the fourth type of identifier moiety can be a same type of identifier moiety.

In some embodiments, the first type of linker, the second type of linker, the third type of linker, and the fourth type of linker can be a same type of linker.

In some embodiments, (c) comprises detecting the identifier moiety while the identifier moiety is associated with a polymerase. In some embodiments, (c) comprises detecting the identifier moiety upon the cleavage of the identifier moiety from the polyphosphate chain and the generation of the growing nucleic acid strand. In some embodiments, (c) comprises detecting the identifier moiety when the identifier moiety is in vicinity of the sensor moiety. In some embodiments, (c) comprises detecting the identifier moiety when the identifier moiety translocates into and through the sensor moiety.

In some embodiments, the time between detection of the identifier moiety and (i) association of the identifier moiety with a polymerase, (ii) cleavage of the identifier moiety, (iii) generation of the growing nucleic acid strand, (iv) bringing the identifier moiety to the vicinity of the sensor moiety, or (v) translocation of the identifier moiety into and through the sensor moiety is at most about 5 minutes (min), at most about 4 min, at most about 3 min, at most about 2 min, at most about 1 min, at most about 50 seconds(s), at most about 40 s, at most about 30 s, at most about 20 s, at most about 10 s, at most about 1 s, at most about 900 milliseconds (ms), at most about 800 ms, at most about 700 ms, at most about 600 ms, at most about 500 ms, at most about 400 ms, at most about 300 ms, at most about 200 ms, at most about 100 ms, at most about 50 ms, at most about 10 ms, at most about 1 ms, at most about 900 microseconds (us), at most about 800 μs, at most about 700 μs, at most about 600 μs, at most about 500 μs, at most about 400 μs, at most about 300 μs, at most about 200 μs, at most about 100 μs, at most about 50 μs, at most about 10 μs, at most about 1 μs, at most about 900 nanoseconds (ns), at most about 800 ns, at most about 700 ns, at most about 600 ns, at most about 500 ns, at most about 400 ns, at most about 300 ns, at most about 200 ns, at most about 100 ns, at most about 90 ns, at most about 80 ns, at most about 70 ns, at most about 60 ns, at most about 50 ns, at most about 40 ns, at most about 30 ns, at most about 20 ns, at most about 10 ns, at most about 9 ns, at most about 8 ns, at most about 7 ns, at most about 6 ns, at most about 5 ns, at most about 4 ns, at most about 3 ns, at most about 2 ns, at most about 1 ns, or less.

In some embodiments, the detection of the identifier moiety is substantially in real-time relative to (i) association of the identifier moiety with a polymerase, (ii) cleavage of the identifier moiety, (iii) generation of the growing nucleic acid strand, (iv) bringing the identifier moiety to the vicinity of the sensor moiety, or (v) translocation of the identifier moiety into and through the sensor moiety. In some embodiments, the detection of the identifier moiety is immediately after or within a short period of time after (i) association of the identifier moiety with a polymerase, (ii) cleavage of the identifier moiety, (iii) generation of the growing nucleic acid strand, (iv) bringing the identifier moiety to the vicinity of the sensor moiety, or (v) translocation of the identifier moiety into and through the sensor moiety. In some embodiments, the short period of time is at most about 1 ms, at most about 900 μs, at most about 800 μs, at most about 700 μs, at most about 600 μs, at most about 500 μs, at most about 400 μs, at most about 300 μs, at most about 200 μs, at most about 100 μs, at most about 50 μs, at most about 10 us, at most about 1 μs, at most about 900 ns, at most about 800 ns, at most about 700 ns, at most about 600 ns, at most about 500 ns, at most about 400 ns, at most about 300 ns, at most about 200 ns, at most about 100 ns, at most about 90 ns, at most about 80 ns, at most about 70 ns, at most about 60 ns, at most about 50 ns, at most about 40 ns, at most about 30 ns, at most about 20 ns, at most about 10 ns, at most about 9 ns, at most about 8 ns, at most about 7 ns, at most about 6 ns, at most about 5 ns, at most about 4 ns, at most about 3 ns, at most about 2 ns, at most about 1 ns, or less.

In an aspect, the present disclosure provides a system for analyzing a target nucleic acid molecule. The system may comprise a sensor moiety configured to detect one or more signals indicative of an electrical property (e.g., capacitance, resistance, impedance, conductivity, voltage, or a change thereof) in the sensor moiety when at least a portion of the target molecule is bound by or in proximity to at least a portion of the sensor moiety. In some cases, the electrical property can be impedance or an impedance change. The one or more signals may be usable to analyze or identify the target molecule.

The system may comprise at least one of the sensor moiety disclosed herein. The system may comprise at least 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1,000 or more sensor moieties. The system may comprise at most about 1,000, at most about 900, at most about 800, at most about 700, at most about 600, at most about 500, at most about 400, at most about 300, at most about 200, at most about 100, at most about 90, at most about 80, at most about 70, at most about 60, at most about 50, at most about 40, at most about 30, at most about 20, at most about 10, at most about 9, at most about 8, at most about 7, at most about 6, at most about 5, at most about 4, at most about 3, at most about 2, or less sensor moieties.

A detected signal indicative of an impedance or impedance change in the sensor moiety induced by the target molecule maybe a single measurement. Alternatively, the detected signal may be a median or average of a plurality of measurements.

When detecting the one or more signals indicative of the impedance or impedance change in the sensor moiety, at least a portion of the target molecule may be bound to a binding moiety of the sensor moiety. The binding moiety may be configured to bind the at least the portion of the target molecule (e.g., a nucleotide, an amino acid, a small molecule, an ion, etc.). The sensor moiety disclosed herein may comprise at least one binding moiety. The sensor moiety may comprise at least 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1,000 or more binding moieties. The sensor moiety may comprise at most about 1,000, at most about 900, at most about 800, at most about 700, at most about 600, at most about 500, at most about 400, at most about 300, at most about 200, at most about 100, at most about 90, at most about 80, at most about 70, at most about 60, at most about 50, at most about 40, at most about 30, at most about 20, at most about 10, at most about 9, at most about 8, at most about 7, at most about 6, at most about 5, at most about 4, at most about 3, at most about 2, or less binding moieties.

In some embodiments, the sensor moiety comprises a pore and/or an enzyme. In some embodiments, the pore is part of a nanopore protein. In some embodiments, the pore is part of a solid-state nanopore. In some embodiments, the enzyme is coupled to the pore. In some embodiments, the enzyme is a polymerase.

In some embodiments, the sensor moiety may be configured to measure the fluorescent signals of the fluorescent labels.

Upon contacting the complex with the engineered nucleotide molecule, the identifier moiety on the engineered nucleotide molecule can interact with the sensor moiety and generate signals. The identifier moiety can detect the identifier moiety while the identifier moiety is associated with a polymerase. Alternatively, the sensor moiety can measure signals upon the cleavage of the identifier moiety from the polyphosphate chain and the generation of the growing nucleic acid strand. In some embodiments, the identifier moiety can be big enough that the sensor moiety can detect its presence when the identifier moiety is in vicinity of the sensor moiety. In some embodiments, the identifier moiety can be detected when it translocates into and through the sensor moiety.

When the engineered nucleotide molecule is added to the growing nucleic acid strand, the protecting group can inhibit coupling of an additional nucleotide to the growing nucleic acid strand. Therefore, the sensor moiety can determine a nucleotide type on the target nucleic acid molecule in one cycle. In some embodiments, the protecting group of the terminal nucleotide molecule on the growing strand may be cleaved, regenerating a hydroxyl group on the nucleotide to allow subsequent addition of another nucleotide molecule. In some embodiments, the protecting group may be cleaved by any suitable reaction, for example, an enzymatic reaction (e.g., by Bacillus stearothermophilus DNA polymerase I), an enzyme-free chemical reaction (e.g., with phosphine, sodium dithionite, palladium catalyzed reaction,), thermal reaction (e.g., in a PCR buffer containing 50 mM KCl, 1.5 mM MgCl₂, 20 mM Tris (pH 8.4 at 25° C.)), or photo cleaving reaction (e.g., upon exposure to an electromagnetic radiation, such as ultraviolet (UV) light).

In some embodiments, the protecting group is cleaved off of the engineered nucleotide molecule at least about 1 ns, at least about 5 ns, at least about 10 ns, at least about 50 ns, at least about 100 ns, at least about 500 ns, at least about 1 μs, at least about 10 μs, at least about 50 μs, at least about 100 μs, at least about 500 μs, at least about 1 ms, at least about 10 ms, at least about 50 ms, at least about 100 ms, at least about 500 ms, at least about 1 s, at least about 10 s, at least about 50 s, at least about 100 s, or more after the engineered nucleotide molecule which the protecting group is attached to is added to the growing nucleic acid strand.

In some embodiments, the protecting group is cleaved off of the engineered nucleotide molecule at least about 1 ns, at least about 5 ns, at least about 10 ns, at least about 50 ns, at least about 100 ns, at least about 500 ns, at least about 1 μs, at least about 10 μs, at least about 50 μs, at least about 100 μs, at least about 500 μs, at least about 1 ms, at least about 10 ms, at least about 50 ms, at least about 100 ms, at least about 500 ms, at least about 1 s, at least about 10 s, at least about 50 s, at least about 100 s, or more after the identifier moiety is detected by the sensor moiety. In some embodiments, the protecting group is cleaved off of the engineered nucleotide molecule when the identifier moiety is being detected by the sensor moiety.

After the hydroxyl group is regenerated, step a) can re-start, thus allowing for the determining of the next nucleotide on the target nucleic acid molecule. This process can be repeated until the sequence of the whole or a desired length of the target nucleic acid molecule is determined.

### II. Sample

In some embodiments, the target nucleic acid molecule can be derived from a sample of interest (e.g., a biological sample from a subject).

Samples for analysis, as disclosed herein, can comprise a plurality of polynucleotides. A polynucleotide can be single stranded DNA, double stranded DNA, or a combination thereof. The polynucleotides can comprise genomic DNA, genomic cDNA, cell free DNA, cell free cDNA, or a combination of any of the foregoing.

A polynucleotide can include cell-free DNA, circulating tumor DNA, genomic DNA, and DNA from formalin fixed and paraffin embedded (FFPE) samples. In some examples, an extracted DNA from a FFPE sample may be damaged, and such damaged DNA may be repaired by an available FFPE DNA repair kit. A sample can comprise any suitable DNA and/or cDNA sample such as for example, urine, stool, blood, saliva, tissue, biopsy, bodily fluid, or tumor cells.

A polynucleotide sample can be derived from any suitable source. For example, a sample can be obtained from a patient, from an animal, from a plant, or from the environment such as, for example, a naturally occurring or artificial atmosphere, a water system, soil, an atmospheric pathogen collection system, a sub-surface sediment, groundwater, or a sewage treatment plant.

Polynucleotides from a sample may include one or more different polynucleotides, such as, for example, DNA, RNA, ribosomal RNA (rRNA), transfer RNA (tRNA), micro RNA (miRNA), messenger RNA (mRNA), fragments of any of foregoing, or combinations of any of the foregoing. A sample can comprise DNA. A sample can comprise genomic DNA. A sample can comprise mitochondrial DNA, chloroplast DNA, plasmid DNA, bacterial artificial chromosomes, yeast artificial chromosomes, oligonucleotide tags, or a combination of any of the foregoing.

The polynucleotides may be single-stranded, double-stranded, or a combination thereof. A polynucleotide can be a single-stranded polynucleotide, which may or may not be in the presence of double-stranded polynucleotides.

The starting amount of polynucleotides in a sample can be, for example, less than about 50 ng, such as less than about 45 ng, less than about 40 ng, less than about 35 ng, less than about 30 ng, less than about 25 ng, less than about 20 ng, less than about 15 ng, less than about 10 ng, less than about 5 ng, less than about 4 ng, less than about 3 ng, less than about 2 ng, less than about 1 ng, less than about 0.5 ng, less than about 0.1 ng, or less. The starting amount of polynucleotides in a sample can be, for example, more than about 0.1 ng, such as more than about 0.5 ng, more than about 1 ng, more than about 2 ng, more than about 3 ng, more than about 4 ng, more than about 5 ng, more than about 10 ng, more than about 15 ng, more than about 20 ng, more than about 25 ng, more than about 30 ng, more than about 35 ng, more than about 40 ng, more than about 45 ng, more than about 50 ng, or more. An amount of starting polynucleotides can be, for example, from about 0.1 ng to about 100 ng, from about 1 ng to about 75 ng, from about 5 ng to about 50 ng, or from about 10 ng to about 20 ng.

The polynucleotides in a sample can be single-stranded, either as obtained or by way of treatment (e.g., denaturation). Polynucleotides can be subjected to subsequent steps (e.g., circularization and amplification) without an extraction step, and/or without a purification step. For example, a fluid sample may be treated to remove cells without an extraction step to produce a purified liquid sample and a cell sample, followed by isolation of the polynucleotides from the purified fluid sample. A variety of procedures for isolation of polynucleotides are available, such as by precipitation or non-specific binding to a substrate followed by washing the substrate to release bound polynucleotides. Where polynucleotides are isolated from a sample without a cellular extraction step, polynucleotides will largely be extracellular or “cell-free” polynucleotides, which may correspond to dead or damaged cells. The identity of such cells may be used to characterize the cells or population of cells from which they are derived, such as in a microbial community.

A sample can be from a subject. A subject can be any suitable organism including, for example, plants, animals, fungi, protists, monerans, viruses, mitochondria, and chloroplasts. Sample polynucleotides can be isolated from a subject, such as a cell sample, tissue sample, bodily fluid sample, or organ sample or cell cultures derived from any of these, including, for example, cultured cell lines, biopsy, blood sample, cheek swab, or fluid sample containing a cell such as saliva. The subject may be an animal such as a cow, a pig, a mouse, a rat, a chicken, a cat, a dog, or a mammal, such as a human. A sample can comprise tumor cells, such as in a sample of tumor tissue from a subject.

Other examples of sample sources may include those from blood, urine, feces, nares, the lungs, the gut, other bodily fluids or excretions, a derivative thereof, or a combination thereof.

A sample from a single individual can be divided into multiple separate samples, such as 2, 3, 4, 5, 6, 7, 8, 9, 10, or more separate samples that are subjected to methods of the disclosure independently, such as analysis in duplicate, triplicate, quadruplicate, or more. Where a sample is from a subject, a reference sequence may also be derived from the subject, such as a consensus sequence from the sample under analysis or the sequence of polynucleotides from another sample or tissue of the same subject. For example, a blood sample may be analyzed for ctDNA mutations, and cellular DNA from another sample from the subject such as a buccal or skin sample, can be analyzed to determine a reference sequence.

Polynucleotides can be extracted from a sample, with or without extraction from cells in a sample, according to any suitable method.

A plurality of polynucleotides can comprise cell-free polynucleotides, such as cell-free DNA (cfDNA) or circulating tumor DNA (ctDNA). Cell-free DNA circulates in both healthy and diseased individuals. cfDNA from tumors (ctDNA) is not confined to any specific cancer type, but appears to be a common finding across different malignancies. The free circulating DNA concentration in plasma can be lower in control subjects in comparison to that in patients having or suspected of having a condition. In an example, the free circulating DNA concentration in plasma can be, for example, from 14 ng/ml to 18 ng/ml in control subjects and from 18 ng/mL to 318 ng/mL in patients with neoplasia.

Apoptotic and necrotic cell death may contribute to cell-free circulating DNA in bodily fluids. For example, significantly increased circulating DNA levels may be observed in plasma of prostate cancer patients and other prostate diseases, such as Benign Prostate Hyperplasia and Prostatits. In addition, circulating tumor DNA may be present in fluids originating from the organs where the primary tumor occurs. In an example, breast cancer detection can be achieved in ductal lavages; colorectal cancer detection in stool; lung cancer detection in sputum, and prostate cancer detection in urine or ejaculate. Cell-free DNA may be obtained from a variety of sources. An example source may be blood samples of a subject. However, cfDNA or other fragmented DNA may be derived from a variety of other sources including, for example, urine and stool samples can be a source of cfDNA, including ctDNA.

### III. Nanopore

A system for analyzing a target nucleic acid molecule can include a reaction chamber that includes one or more nanopore devices. A nanopore device may be an individually addressable nanopore device. An individually addressable nanopore can be individually readable. An individually addressable nanopore can be individually writable. An individually addressable nanopore can be individually readable and individually writable. The system can include one or more computer processors for facilitating sample preparation and various operations of the disclosure, such as polynucleotide sequencing. The processor can be coupled to nanopore device.

A nanopore device may include a plurality of individually addressable sensing electrodes. Each sensing electrode can include a membrane adjacent to the electrode, and one or more nanopores in the membrane. A nanopore may be in a membrane such as a lipid bi-layer disposed adjacent or in sensing proximity to an electrode that is part of, or coupled to, an integrated circuit. A nanopore may be associated with an individual electrode and sensing integrated circuit or a plurality of electrodes and sensing integrated circuits. A nanopore can comprise a solid state nanopore. A nanopore device may include a reference electrode.

In some cases, the sensor moiety may be configured to detect one or more signals indicative of the impedance or impedance change, e.g., between a sensing electrode and a reference electrode, when at least a portion of an engineered nucleotide molecule is bound to at least a portion of the sensor moiety, e.g., the sensing electrode. Alternatively, the sensor moiety may be configured to detect one or more signals indicative of the impedance or impedance change, e.g., between the sensing electrode and the reference electrode, when at least a portion of an engineered nucleotide molecule is not bound but in proximity to at least a portion of the sensor moiety, e.g., the sensing electrode.

The sensor moiety of the present disclosure may be configured to detect more signals indicative of the impedance or impedance change, e.g., between the sensing electrode and the reference electrode, when a distance between (i) at least a portion of an engineered nucleotide molecule and (ii) the sensing electrode is at least about 0.1 nm, at least about 0.5 nm, at least about 1 nm, at least about 2 nm, at least about 3 nm, at least about 4 nm, at least about 5 nm, at least about 6 nm, at least about 7 nm, at least about 8 nm, at least about 9 nm, at least about 10 nm, at least about 20 nm, at least about 30 nm, at least about 40 nm, at least about 50 nm, at least about 60 nm, at least about 70 nm, at least about 80 nm, at least about 90 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1,000 μm, or more. The sensor moiety as disclosed herein may be configured to detect more signals indicative of the impedance or impedance change, e.g., between the sensing electrode and the reference electrode, when a distance between (i) at least a portion of an engineered nucleotide molecule and (ii) the sensing electrode is at most about 1,000 μm, at most about 900 μm, at most about 800 μm, at most about 700 μm, at most about 600 μm, at most about 500 μm, at most about 400 μm, at most about 300 μm, at most about 200 μm, at most about 100 μm, at most about 90 μm, at most about 80 μm, at most about 70 μm, at most about 60 μm, at most about 50 μm, at most about 40 μm, at most about 30 μm, at most about 20 μm, at most about 10 μm, at most about 9 μm, at most about 8 μm, at most about 7 μm, at most about 6 μm, at most about 5 μm, at most about 4 μm, at most about 3 μm, at most about 2 μm, at most about 1 μm, at most about 900 nm, at most about 800 nm, at most about 700 nm, at most about 600 nm, at most about 500 nm, at most about 400 nm, at most about 300 nm, at most about 200 nm, at most about 100 nm, at most about 90 nm, at most about 80 nm, at most about 70 nm, at most about 60 nm, at most about 50 nm, at most about 40 nm, at most about 30 nm, at most about 20 nm, at most about 10 nm, at most about 9 nm, at most about 8 nm, at most about 7 nm, at most about 6 nm, at most about 5 nm, at most about 4 nm, at most about 3 nm, at most about 2 nm, at most about 1 nm, at most about 0.5 nm, at most about 0.1 nm, or less.

The sensor moiety of the present disclosure may be configured to detect more signals indicative of the impedance or impedance change, e.g., between the sensing electrode and the reference electrode, when an engineered nucleotide molecule is within a predetermined space that is near or adjacent to the sensing electrode. The predetermined space may be characterized by having a volume of at least about 0.1 nm², at least about 0.5 nm², at least about 1 nm, at least about 2 nm², at least about 3 nm², at least about 4 nm², at least about 5 nm², at least about 6 nm², at least about 7 nm², at least about 8 nm², at least about 9 nm², at least about 10 nm², at least about 20 nm², at least about 30 nm², at least about 40 nm², at least about 50 nm², at least about 60 nm², at least about 70 nm², at least about 80 nm², at least about 90 nm², at least about 100 nm², at least about 200 nm², at least about 300 nm², at least about 400 nm², at least about 500 nm², at least about 600 nm², at least about 700 nm², at least about 800 nm², at least about 900 nm², at least about 1 μm², at least about 2 μm², at least about 3 μm², at least about 4 μm², at least about 5 μm², at least about 6 μm², at least about 7 μm², at least about 8 μm², at least about 9 μm², at least about 10 μm², at least about 20 μm², at least about 30 μm², at least about 40 μm², at least about 50 μm², at least about 60 μm², at least about 70 μm², at least about 80 μm², at least about 90 μm², at least about 100 μm², at least about 200 μm², at least about 300 μm², at least about 400 μm², at least about 500 μm², at least about 600 μm², at least about 700 μm², at least about 800 μm², at least about 900 μm², at least about 1,000 μm², or more. The predetermined space may be characterized by having a volume of at most about 1,000 μm², at most about 900 μm², at most about 800 μm², at most about 700 μm², at most about 600 μm², at most about 500 μm², at most about 400 μm², at most about 300 μm², at most about 200 μm², at most about 100 μm², at most about 90 μm², at most about 80 μm², at most about 70 μm², at most about 60 μm², at most about 50 μm², at most about 40 μm², at most about 30 μm², at most about 20 μm², at most about 10 μm², at most about 9 μm², at most about 8 μm², at most about 7 μm², at most about 6 μm², at most about 5 μm², at most about 4 μm², at most about 3 μm², at most about 2 μm², at most about 1 μm², at most about 900 nm², at most about 800 nm², at most about 700 nm², at most about 600 nm², at most about 500 nm², at most about 400 nm², at most about 300 nm², at most about 200 nm², at most about 100 nm², at most about 90 nm², at most about 80 nm², at most about 70 nm², at most about 60 nm², at most about 50 nm², at most about 40 nm², at most about 30 nm², at most about 20 nm², at most about 10 nm², at most about 9 nm², at most about 8 nm², at most about 7 nm², at most about 6 nm², at most about 5 nm², at most about 4 nm², at most about 3 nm², at most about 2 nm², at most about 1 nm², at most about 0.5 nm², at most about 0.1 nm², or less.

Devices and systems for use in methods provided by the present disclosure may accurately detect individual nucleotide incorporation events, such as upon the incorporation of a nucleotide into a growing strand that is complementary to a template. An enzyme such as a DNA polymerase, RNA polymerase, and/or ligase can participate in incorporation of nucleotides to a growing polynucleotide chain. Enzymes such as polymerases can generate polynucleotide strands.

The added nucleotide can be complimentary to the corresponding template polynucleotide strand which is hybridized to the growing strand. A nucleotide can include a tag or tag species that is coupled to any location of the nucleotide including, but not limited to a phosphate such as a γ-phosphate, sugar or nitrogenous base moiety of the nucleotide. In some cases, tags are detected while tags are associated with a polymerase during the incorporation of nucleotide tags. The tag may continue to be detected until the tag translocates through the nanopore after nucleotide incorporation and subsequent cleavage and/or release of the tag. Nucleotide incorporation events can release tags from the nucleotides which pass through a nanopore and are detected. A tag can be released by the polymerase, or cleaved/released in any suitable manner including without limitation cleavage by an enzyme located near the polymerase. In this way, the incorporated base may be identified (i.e., A, C, G, T or U) because a unique tag is released from each type of nucleotide (i.e., adenine, cytosine, guanine, thymine or uracil). In nucleotide incorporation events that do not release, a tag coupled to an incorporated nucleotide is detected with the aid of a nanopore. In some examples, the tag can move through or in proximity to the nanopore and be detected with the aid of the nanopore.

Methods and systems of the disclosure can enable the detection of polynucleotide incorporation events, such as at a resolution of at least 1, at least about 2, at least about 3, at least about 4, at least about 5, at least about 6, at least about 7, at least about 8, at least about 9, at least about 10, at least about 20, at least about 30, at least about 40, at least about 50, at least about 100, at least about 500, at least about 1000, at least about 5000, at least about 10000, at least about 50000, at least about 100000 or more polynucleotide bases within a given time period. For example, a nanopore device can be used to detect individual polynucleotide incorporation events, with each event being associated with an individual nucleic acid base. In other examples, a nanopore device can be used to detect an event that is associated with a plurality of bases. For example, a signal sensed by the nanopore device can be a combined signal from at least about 2, at least about 3, at least about 4, or at least about 5 bases.

In certain sequencing methods, tags do not pass through the nanopore. The tags can be detected by the nanopore and exit the nanopore without passing through the nanopore such as exiting from the inverse direction from which the tag entered the nanopore. A sequencing device can be configured to actively expel the tags from the nanopore.

In certain sequencing methods tags are not released upon nucleotide incorporation events. Nucleotide incorporation events can present tags to a nanopore without releasing the tags. The tags can be detected by the nanopore without being released. The tags may be attached to the nucleotides by a linker of sufficient length to present the tag to the nanopore for detection.

In some embodiments, the nucleic acid can be sequenced with sequential addition and/or removal of the engineered nucleotide molecule.

Nucleotide incorporation events may be detected in real-time as they occur by a nanopore. An enzyme such as a DNA polymerase attached to or in proximity to a nanopore can facilitate the flow of a polynucleotide through or adjacent to a nanopore. A nucleotide incorporation event, or the incorporation of a plurality of nucleotides, may release or present one or more tags, which may be detected by a nanopore. Detection can occur as the tags flow through or adjacent to the nanopore, as the tags reside in the nanopore and/or as the tags are presented to the nanopore. In some cases, an enzyme attached to or in proximity to the nanopore may aid in detecting tags upon the incorporation of one or more nucleotides.

The nanopore may be formed or otherwise embedded in a membrane disposed adjacent to a sensing electrode of a sensing circuit, such as an integrated circuit. An integrated circuit may be an application specific integrated circuit (ASIC). An integrated circuit can be a field effect transistor or a complementary metal-oxide semiconductor (CMOS). A sensing circuit may be situated in a chip or other device having the nanopore, or off of the chip or device, such as in an off-chip configuration.

As a nucleic acid or tag flows through or adjacent to the nanopore, the sensing circuit detects an electrical signal associated with the nucleic acid or tag. The nucleic acid may be a subunit of a larger strand. The tag may be a byproduct of a nucleotide incorporation event or other interaction between a tagged nucleic acid and the nanopore or a species adjacent to the nanopore, such as an enzyme that cleaves a tag from a nucleic acid. The tag may remain attached to the nucleotide. A detected signal may be collected and stored in a memory location, and later used to construct a sequence of the nucleic acid. The collected signal may be processed to account for any abnormalities in the detected signal, such as errors.

Nanopores may be used to sequence polynucleotides indirectly, in some cases with electrical detection. Indirect sequencing may be any method where an incorporated nucleotide in a growing strand does not pass through the nanopore. The polynucleotide may pass within any suitable distance from and/or proximity to the nanopore, in some cases within a distance such that tags released from nucleotide incorporation events are detected in the nanopore.

Byproducts of nucleotide incorporation events may be detected by the nanopore. Nucleotide incorporation events refer to the incorporation of a nucleotide into a growing polynucleotide chain. A byproduct may be correlated with the incorporation of a given type nucleotide. Nucleotide incorporation events can be catalyzed by an enzyme, such as DNA polymerase, and use base pair interactions with a template molecule to choose amongst the available nucleotides for incorporation at each location.

A nucleic acid sample may be sequenced using tagged nucleotides or nucleotide analogs. In some examples, a method for sequencing a nucleic acid molecule comprises (a) incorporating (e.g., polymerizing) tagged nucleotides, wherein a tag associated with an individual nucleotide is released upon incorporation, and (b) detecting the released tag with the aid of a nanopore. In some instances, the method further comprises directing the tag attached to or released from an individual nucleotide through the nanopore. The released or attached tag may be directed by any suitable technique, in some cases with the aid of an enzyme (or molecular motor) and/or a voltage difference across the pore. Alternative, the released or attached tag may be directed through the nanopore without the use of an enzyme. For example, the tag may be directed by a voltage difference across the nanopore as described herein.

A tag may be detected with the aid of a nanopore device having at least one nanopore in a membrane. The tag may be associated with an individual tagged nucleotide during incorporation of the individual tagged nucleotide. A nanopore device can detect a tag associated with an individual tagged nucleotide during incorporation. The tagged nucleotides, whether incorporated into a growing nucleic acid strand or unincorporated, can be detected, determined, or differentiated for a given period of time by the nanopore device, in some cases with the aid of an electrode and/or nanopore of the nanopore device. The time period within which the nanopore device detects the tag may be shorter, in some cases substantially shorter, than the time period in which the tag and/or nucleotide coupled to the tag is held by an enzyme, such as an enzyme facilitating the incorporation of the nucleotide into a nucleic acid strand (e.g., a polymerase). A tag can be detected by the electrode a plurality of times within the time period that the incorporated tagged nucleotide is associated with the enzyme. For instance, the tag can be detected by the electrode at least 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 200, at least about 300, at least about 400, at least about 500, at least about 1000, at least about 10,000, at least about 100,000, or at least about 1,000,000 times within the time period that the incorporated tagged nucleotide is associated with the enzyme.

A tag associated with an individual nucleotide can be detected by a nanopore without being released from the nucleotide upon incorporation. Tags can be detected without being released from incorporated nucleotides during synthesis of a nucleic acid strand that is complementary to a target strand. The tags can be attached to the nucleotides with a linker such that the tag is presented to the nanopore (e.g., the tag hangs down into or otherwise extend through at least a portion of the nanopore). The length of the linker may be sufficiently long so as to permit the tag to extend to or through at least a portion of the nanopore. In some instances, the tag is presented to (i.e., moved into) the nanopore by a voltage difference. Other ways to present the tag into the pore may also be suitable (e.g., use of enzymes, magnets, electric fields, pressure differential). In some instances, no active force is applied to the tag (i.e., the tag diffuses into the nanopore).

A chip for sequencing a nucleic acid sample can comprise a plurality of individually addressable nanopores. An individually addressable nanopore of the plurality can contain at least one nanopore formed in a membrane disposed adjacent to an integrated circuit. Each individually addressable nanopore can be capable of detecting a tag associated with an individual nucleotide. The nucleotide can be incorporated (e.g., polymerized) and the tag may not be released from the nucleotide upon incorporation.

Tags can be presented to the nanopore upon nucleotide incorporation events and are released from the nucleotide. The released tags can go through the nanopore. The tags do not pass through the nanopore in some instances. A tag that has been released upon a nucleotide incorporation event is distinguished from a tag that may flow through the nanopore, but has not been released upon a nucleotide incorporation event at least in part by the dwell time in the nanopore. In some cases, tags that dwell in the nanopore for at least 100 milliseconds (ms) are released upon nucleotide incorporation events and tags that dwell in the nanopore for less than 100 ms are not released upon nucleotide incorporation events. Tags may be captured and/or guided through the nanopore by a second enzyme or protein (e.g., a nucleic acid binding protein). The second enzyme may cleave a tag upon (e.g., during or after) nucleotide incorporation. A linker between the tag and the nucleotide may be cleaved.

Incorporated nucleic acids can be detected by and/or are detectable by the nanopore for a shorter period of time than an un-incorporated nucleotide. Alternatively, incorporated nucleic acids can be detected by and/or are detectable by the nanopore for a longer period of time than an un-incorporated nucleotide. The difference and/or ratio between these times can be used to determine whether a nucleotide detected by the nanopore is incorporated or not, as described herein.

The detection period can be based on the free-flow of the nucleotide through the nanopore; an unincorporated nucleotide may dwell at or in proximity to the nanopore for a time period between 1 nanosecond (ns) and 100 ms, or between 1 ns and 50 ms, whereas an incorporated nucleotide may dwell at or in proximity to the nanopore for a time between 50 ms and 500 ms, or 100 ms and 200 ms. The time periods can vary based on processing conditions; however, an incorporated nucleotide may have a dwell time that is greater than that of an unincorporated nucleotide.

### IV. Polymerase

A DNA polymerase can be bound to the 3′ end of a gap of the nucleic acid (NA) molecule as disclosed herein (e.g., the 3′ end of a heterologous gap of the circularized NA molecule). DNA sequencing can be accomplished by using an enzyme such as a DNA polymerize to amplify and transcribe a polynucleotide in proximity to a nanopore and tagged nucleotides. Sequencing methods can involve incorporating or polymerizing tagged nucleotides using a polymerase such as a DNA polymerase, or transcriptase. The polymerase can be mutated to allow it to accept tagged nucleotides. The polymerase can also be mutated to increase the time for which the tag is detected by the nanopore.

A sequencing enzyme can be, for example, any suitable enzyme that creates a polynucleotide strand by phosphate linkage of nucleotides. The DNA polymerase can be, for example, a 9° Nm™ polymerase or a variant thereof, an E. Coli DNA polymerase I, a Bacteriophage T4 DNA polymerase, a Sequenase, a Taq DNA polymerase, a 9° Nm™ polymerase (exo-) A485L/Y409V, a ϕ29 DNA Polymerase, a Bst DNA polymerase, or variants, mutants, or homologs of any of the foregoing. A homolog can have any suitable percentage homology such as, for example, at least 30%, at least 40%, at least 50%, at least 60%, at least 70%, at least 80%, at least 90%, or at least 95% sequence identity.

In some examples, for nanopore sequencing, a polymerization enzyme can be attached to or situated in proximity to a nanopore. Suitable methods for attaching the polymerization enzyme to a nanopore include cross-linking the enzyme to the nanopore or in proximity to the nanopore such as via the formation of intra-molecular disulfide bonds. The nanopore and the enzyme may also be a fusion such as an encoded by a single polypeptide chain. Methods for producing fusion proteins may include fusing the coding sequence for the enzyme in frame and adjacent to the coding sequence for the nanopore and expressing this fusion sequence from a single promoter. A polymerization enzyme can be attached or coupled to a nanopore using molecular staples or protein fingers. A polymerization enzyme can be attached to a nanopore via an intermediate molecule, such as for example biotin conjugated to both the enzyme and the nanopore with streptavidin tetramers linked to both biotins. The intermediate molecule can be referred to as a linker.

The sequencing enzyme can also be attached to a nanopore with an antibody. Proteins that form a covalent bond between each other can be used to attach a polymerase to a nanopore. Phosphatase enzymes or an enzyme that cleaves a tag from a nucleotide can also be attached to the nanopore.

The polymerase can be mutated to facilitate and/or to improve the efficiency of the mutated polymerase for incorporation of tagged nucleotides into a growing polynucleotide relative to the non-mutated polymerase. The polymerase can be mutated to improve entry of the nucleotide analog such as a tagged nucleotide, into the active site region of the polymerase and/or mutated for coordinating with the nucleotide analogs in the active region.

Other mutations such as amino acid substitutions, insertions, deletions, and/or exogenous features to a polymerize can result in enhanced metal ion coordination, reduced exonuclease activity, reduced reaction rates at one or more steps of the polymerase kinetic cycle, decreased branching fraction, altered cofactor selectivity, increased yield, increased thermostability, increased accuracy, increased speed, increased read length, increased salt tolerance relative to the non-mutated polymerase.

A suitable polymerase can have a kinetic rate profile that is suitable for detection of the tags by a nanopore. The rate profile generally refers to the overall rate of nucleotide incorporation and/or a rate of any step of nucleotide incorporation such as nucleotide addition, enzymatic isomerization such as to or from a closed state, cofactor binding or release, product release, incorporation of polynucleotide into the growing polynucleotide, or translocation.

A polymerase can be adapted to permit the detection of sequencing events. The rate profile of a polymerase can be such that a tag is loaded into (and/or detected by) the nanopore for an average of 0.1 milliseconds (ms), 1 ms, 5 ms 10 ms, 20 ms, 30 ms, 40 ms, 50 ms, 60 ms, 80 ms, 100 ms, 120 ms, 140 ms, 160 ms, 180 ms, 200 ms, 220 ms, 240 ms, 260 ms, 280 ms, 300 ms, 400 ms, 500 ms, 600 ms, 800 ms, or 1000 ms. For example, the rate profile of a polymerase can be such that a tag is loaded into and/or detected by the nanopore for an average of at least 5 ms, at least 10 ms, at least 20 ms, at least 30 ms, at least 40 ms, at least 50 ms, at least 60 ms, at least 80 ms, at least 100 ms, at least 120 ms, at least 140 ms, at least 160 ms, at least 180 ms, at least 200 ms, at least 220 ms, at least 240 ms, at least 260 ms, at least 280 ms, at least 300 ms, at least 400 ms, at least 500 ms, at least 600 ms, at least 800 ms, or at least 1000 ms. A tag can be detected by the nanopore for an average between 80 ms and 260 ms, between 100 ms and 200 ms, or between 100 ms and 150 ms.

A nanopore/polymerase complex can be configured to permit the detection of one or more events associated with amplification and transcription of the circular polynucleotide. The one or more events may be kinetically observable and/or non-kinetically observable such as a nucleotide migrating through a nanopore without coming in contact with a polymerase.

In some cases, the polymerase reaction exhibits two kinetic steps which proceed from an intermediate in which a nucleotide or a polyphosphate moiety is bound to the polymerase enzyme, and two kinetic steps which proceed from an intermediate in which the nucleotide and the polyphosphate moiety are not bound to the polymerase enzyme. The two kinetic steps can include enzyme isomerization, nucleotide incorporation, and product release. In some cases, the two kinetic steps are template translocation and nucleotide binding.

A suitable polymerase can exhibit strong or enhanced strand displacement.

### V. Identification of Sequence Variants

Methods provided by the present disclosure can be used to identify sequence variants in a polynucleotide sample. A sequence difference between sequencing reads and a reference sequence is referred to as a genuine sequence variant if the sequence difference occurs in at least two different polynucleotides, e.g., two different circular polynucleotides, which can be distinguished as a result of having different junctions. Because the position and type of a sequence variant that are the result of amplification or sequencing errors are unlikely to be duplicated exactly on two different polynucleotides comprising the same target sequence, including this validation parameter can reduce the background of erroneous sequence variants, with a concurrent increase in the sensitivity and accuracy of detecting actual sequence variation in a sample. A sequence variant can have a frequency less than 5%, less than 4%, less than 3%, less than 2%, less than 1.5%, less than 1%, less than 0.75%, less than 0.5%, less than 0.25%, less than 0.1%, less than 0.075%, less than 0.05%, less than 0.04%, less than 0.03%, less than 0.02%, less than 0.01%, less than 0.005%, less than 0.001%, or lower is sufficiently above background to permit an accurate identification. A sequence variant can occur with a frequency of less than 0.1%. The frequency of a sequence variant can be sufficiently above background when such frequency is statistically significantly above the background error rate, for example, with a p-value less than 0.05, less than 0.01, less than 0.001, or less than 0.0001. The frequency of a sequence variant can be sufficiently above background when the frequency is at least 2-fold, at least 3-fold, at least 4-fold, at least 5-fold, at least 6-fold, at least 7-fold, at least 8-fold, at least 9-fold, at least 10-fold, at least 25-fold, at least 50-fold, at least 100-fold, or more above the background error rate. The background error rate for accurately determining the sequence at a given position can be less than 1%, less than 0.5%, less than 0.1%, less than 0.05%, less than 0.01%, less than 0.005%, less than 0.001%, or less than 0.0005%.

Identifying a sequence variant can comprise optimally aligning one or more sequencing reads with a reference sequence to identify differences between the two, as well as to identify junctions. Alignment can involve placing one sequence along another sequence, iteratively introducing gaps along each sequence, scoring how well the two sequences match, and repeating for various positions along the reference. The best-scoring match is deemed to be the alignment and represents an inference about the degree of relationship between the sequences.

A reference sequence to which sequencing reads are compared is a reference genome, such as the genome of a member of the same species as the subject. A reference genome may be complete or incomplete. A reference genome can consist only of regions containing target polynucleotides, such as from a reference genome or from a consensus generated from sequencing reads under analysis. A reference sequence can comprise or can consist of sequences of polynucleotides of one or more organisms, such as sequences from one or more bacteria, archaea, viruses, protists, fungi, or other organism. A reference sequence can consist of only a portion of a reference genome, such as regions corresponding to one or more target sequences under analysis. For example, for detection of a pathogen, a reference genome can be the entire genome of the pathogen, or a portion thereof useful in identification, such as of a particular strain or serotype. A sequencing read can be aligned to multiple different reference sequences, such as to screen for multiple different organisms or strains.

### VI. Therapeutic Applications

Methods, systems, and compositions provided herein can be directed to one or more therapeutic applications, such as in the characterization of a patient sample and optionally diagnosis of a condition of a subject. Therapeutic applications can include informing the selection of therapies to which a patient may be most responsive and/or treatment of a subject in need of therapeutic intervention based on the results of methods provided by the present disclosure.

For example, methods provided by the present disclosure can be used to diagnose tumor presence, progression and/or metastasis of tumors, such as when the polynucleotides analyzed comprise or consist of cfDNA, ctDNA, or fragmented tumor DNA. A subject may be monitored for tumor treatment efficacy, for example, by monitoring ctDNA over time, a decrease in ctDNA can be used as an indication of treatment efficacy, and increases in ctDNA can inform selection of different treatments and/or different dosages. Other uses include evaluations of organ rejection in transplant recipients such as where increases in the amount of circulating DNA corresponding to the transplant donor genome is used as an early indicator of transplant rejection, and genotyping/isotyping of pathogen infections, such as viral or bacterial infections. Detection of sequence variants in circulating fetal DNA may be used to diagnose a condition of a fetus.

Methods provided by the present disclosure can comprise diagnosing a subject based on a result of the sequencing, such as diagnosing the subject with a disease associated with a detected causal genetic variant, or reporting a likelihood that the patient has or will develop such disease.

A causal genetic variant can include sequence variants associated with a particular type or stage of cancer, or of cancer having a particular characteristic such as metastatic potential, drug resistance, and/or drug responsiveness. Methods provided by the present disclosure can be used to inform therapeutic decisions, guidance and monitoring, of cancer therapies. For example, treatment efficacy can be monitored by comparing patient ctDNA samples from before, during, and after treatment with particular including molecular targeted therapies such as monoclonal drugs, chemotherapeutic drugs, radiation protocols, and combinations of any of the foregoing. For example, the ctDNA can be monitored to see if certain mutations increase or decrease, or new mutations appear, after treatment, which can allow a physician to modify a treatment in a much shorter period of time than afforded by methods of monitoring that track patient symptoms. Methods can comprise diagnosing a subject based on the results of polynucleotide sequencing, such as diagnosing the subject with a particular stage or type of cancer associated with a detected sequence variant, or reporting a likelihood that the patient has or will develop such cancer.

For example, for therapies that are specifically targeted to patients on the basis of molecular markers, patients can be tested to find out if certain mutations are present in their tumor, and these mutations can be used to predict response or resistance to the therapy and guide the decision whether to use the therapy. Detecting and monitoring ctDNA during the course of treatment can be useful in guiding treatment selections.

Sequence variants associated with one or more kinds of cancer that may be used for diagnosis, prognosis, or treatment decisions. For example, suitable target sequences of oncological significance include alterations in the TP53 gene, the ALK gene, the KRAS gene, the PIK3CA gene, the BRAF gene, the EGFR gene, and the KIT gene. A target sequence may be specifically amplified, and/or specifically analyzed for sequence variants may be all or part of a cancer-associated gene.

Methods provided by the present disclosure can be useful in discovering new, rare mutations that are associated with one or more cancer types, stages, or cancer characteristics. For example, in populations of individuals sharing a characteristic under analysis such as a particular disease, type of cancer, and/or stage of cancer, using methods provided by the present disclosure sequence variants can be identified reflecting mutations in particular genes or parts of genes. Identified sequence variants occurring with a statistically significantly greater frequency among the group of individuals sharing the characteristic than in individuals without the characteristic may be assigned a degree of association with that characteristic. The sequence variants or types of sequence variants so identified may then be used in diagnosing or treating individuals discovered to harbor them.

Additional therapeutic applications can include use in non-invasive fetal diagnostics. Fetal DNA can be found in the blood of a pregnant woman. Methods provided by the present disclosure can be used to identify sequence variants in circulating fetal DNA, and thus may be used to diagnose one or more genetic diseases in the fetus, such as those associated with one or more causal genetic variants. Examples of causal genetic variants include trisomies, cystic fibrosis, sickle-cell anemia, and Tay-Saks disease. The mother may provide a control sample and a blood sample to be used for comparison. The control sample may be any suitable tissue, and can then be sequenced to provide a reference sequence. Sequences of cfDNA corresponding to fetal genomic DNA can then be identified as sequence variants relative to the maternal reference. The father may also provide a reference sample to aid in identifying fetal sequences, and sequence variants.

Different therapeutic applications can include detection of exogenous polynucleotides, including from pathogens such as bacteria, viruses, fungi, and microbes, which information may inform a treatment.

### VII. Computer Systems

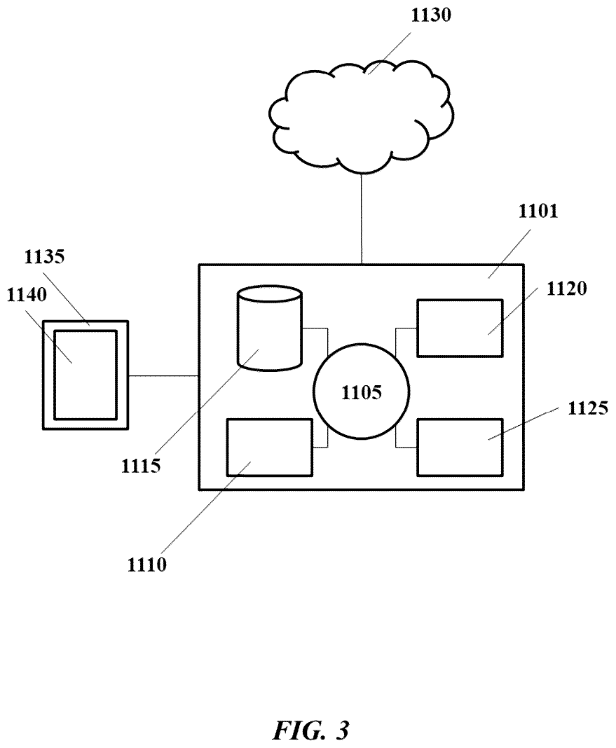

The present disclosure provides computer systems that are programmed to implement methods of the disclosure. FIG. 3 shows a computer system 1101 that is programmed or otherwise configured to communicate with and regulate various aspects of sequencing of the present disclosure. The computer system 1101 can regulate various operations of the sensor moiety, such as detecting one or more signals indicative of an impedance or impedance change in the sensor moiety when at least a portion of a target nucleic acid molecule is bound by a binding moiety of the sensor moiety. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, (i) progress of sequencing, and (ii) sequencing information obtained from sequencing. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, determine sequence readout of a target nucleic acid.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

### EXAMPLES

### Example 1

### 3′-O-azidomethyl-deoxyadenine-5′-hexaphosphate-T40 (3′-O-N₃-dA6P-T40)

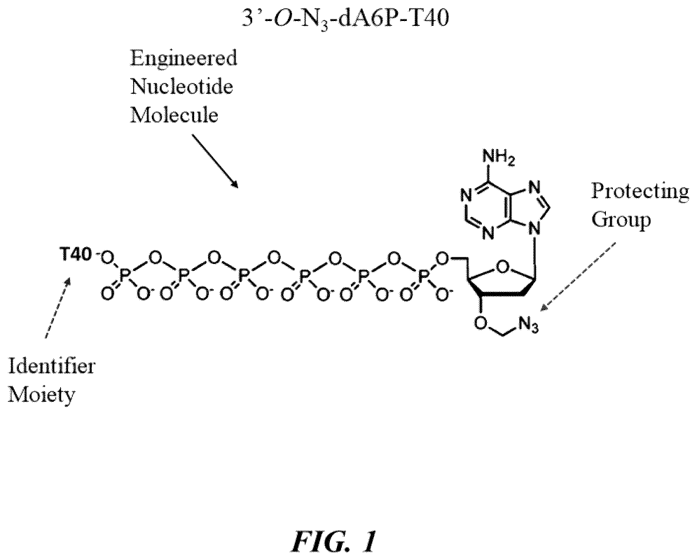

FIG. 1 schematically illustrates an example of an engineered nucleotide molecule as disclosed herein. The engineered nucleotide molecule 3′-O-N₃-dA6P-T40 has a deoxyribose sugar, an A base coupled to the sugar, a hexaphosphate coupled to the sugar, a T40 coupled to the sugar via the hexaphosphate, and an azidomethyl coupled to the 3′-O of the sugar. The molecule shown in FIG. 1 can be used in any methods of sequencing disclosed herein.

### Example 2

### 3′-O-allyl-deoxycytidine-5′-triphosphate-T40 (3′-O-allyl-dCTP-T40)

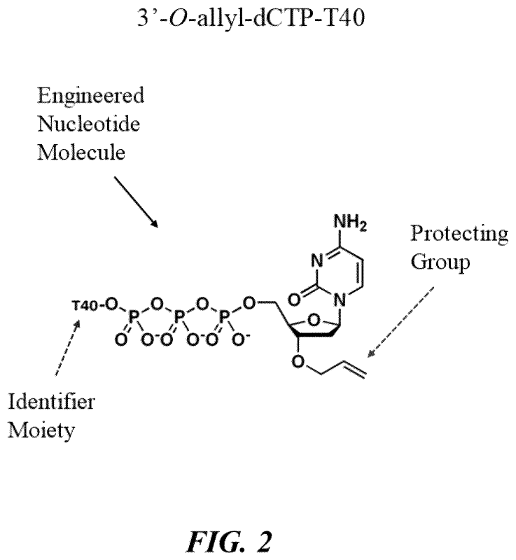

FIG. 2 schematically illustrates an example of an engineered nucleotide molecule as disclosed herein. The engineered nucleotide molecule 3′-O-allyl-dCTP-T40 has a deoxyribose sugar, a C base coupled to the sugar, a triphosphate coupled to the sugar, a T40 coupled to the sugar via the triphosphate, and an allyl coupled to the 3′-O of the sugar. The molecule shown in FIG. 2 can be used in any methods of sequencing disclosed herein.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

1-46. (canceled)

47. An engineered nucleotide molecule, comprising:
a pentose sugar;
a base coupled to said pentose sugar, wherein said base is selected from the group consisting of adenine, guanine, cytosine, thymine, uracil, and an analogue thereof;
a polyphosphate chain coupled to said pentose sugar, wherein said polyphosphate chain comprises two or more phosphate groups; and
an identifier moiety coupled to said pentose sugar, wherein said identifier moiety is specific for said engineered nucleotide molecule, wherein said identifier moiety is coupled to said polyphosphate chain,
wherein said identifier moiety comprises (1) a first monomeric unit and a second monomeric unit, wherein said first monomeric unit is different from said second monomeric unit, or (2) at least one nucleotide molecule, wherein a base of said at least one nucleotide molecule is different from said base of said engineered nucleotide molecule.

48. The engineered nucleotide molecule of claim 47, wherein said pentose sugar is deoxyribose.

49. The engineered nucleotide molecule of claim 47, wherein said polyphosphate chain comprises three or more phosphate groups.

50. The engineered nucleotide molecule of claim 47, further comprising a protecting group coupled to a hydroxyl group of said pentose sugar, wherein said protecting group is configured to inhibit coupling of an additional nucleotide to said engineered nucleotide molecule.

51. The engineered nucleotide molecule of claim 50, wherein said hydroxyl group is at the 3′ position of said pentose sugar, wherein said protecting group comprises allyl or azide.

52. The engineered nucleotide molecule of claim 50, wherein said protecting group is removable from said engineered nucleotide molecule.

53. The engineered nucleotide molecule of claim 47, wherein said identifier moiety is removable from said engineered nucleotide molecule.

54. The engineered nucleotide molecule of claim 47, wherein said identifier moiety comprises said first monomeric unit and said second monomeric unit, and wherein said first monomeric unit comprises a different structure from said second monomeric unit.

55. The engineered nucleotide molecule of claim 54, wherein said identifier moiety comprises a first nucleotide molecule and a second nucleotide molecule, wherein said second nucleotide molecule comprises a different base from said first nucleotide molecule.

56. The engineered nucleotide molecule of claim 54, wherein said first monomeric unit is a nucleotide molecule and said second monomeric unit is a non-nucleotide molecule.

57. The engineered nucleotide molecule of claim 47, wherein said identifier moiety comprises said at least one nucleotide molecule and wherein said base of said at least one nucleotide molecule comprises a different structure from said base of said engineered nucleotide molecule.

58. The engineered nucleotide molecule of claim 47, wherein said identifier moiety comprises a polynucleotide.

59. The engineered nucleotide molecule of claim 58, wherein said polynucleotide has a length of at least about 10 bases.

60. The engineered nucleotide molecule of claim 58, wherein said polynucleotide has a length of at least about 20 bases.

61. The engineered nucleotide molecule of claim 58, wherein said polynucleotide comprises a polyN selected from the group consisting of polyA, polyT, polyC, polyG, polyU, and a variant thereof.

62. The engineered nucleotide molecule of claim 47, wherein said identifier moiety comprises a first polyN and a second polyN, wherein said first polyN comprises a nucleotide that is different from a nucleotide of said second polyN.

63. The engineered nucleotide molecule of claim 62, wherein said nucleotide of said first polyN comprises a different base from said nucleotide of said second polyN.

64. The engineered nucleotide molecule of claim 47, wherein said identifier moiety is configured to induce a change in one or more electrochemical properties of an electrochemical cell when said engineered nucleotide molecule is brought in vicinity of a sensor moiety of said electrochemical cell.

65. The engineered nucleotide molecule of claim 47, wherein said identifier moiety is coupled to said polyphosphate chain via a linker moiety.

66. The engineered nucleotide molecule of claim 65, wherein said linker moiety comprises an ester, ether, thioether, ethylene glycol, alkylene, alkenylene, alkynylene, heteroalkylene, cycloalkylene, heterocyclylene, arylene, heteroarylene, or heterocycloalkylene group.
